(19) United States
(12) Patent Application Publication
(10) Pub. No.: US 2016/0297860 A1
(43) Pub. Date: Oct. 13, 2016

(54) IDENTIFICATION OF A NOVEL B CELL CYTOKINE

(71) Applicant: THE REGENTS OF THE UNIVERSITY OF CALIFORNIA, Oakland, CA (US)

(72) Inventors: Albert Zlotnik, San Diego, CA (US); Peter Hevezi, Encinitas, CA (US); Van Phi Luu, Tustin, CA (US); Amanda M. Burkhardt, Long Beach, CA (US); Irina Ushach, Newport Beach, CA (US)

(73) Assignee: The Regents of the University of California, Oakland, CA (US)

(21) Appl. No.: 15/036,207

(22) PCT Filed: Nov. 20, 2014
(86) PCT No.: PCT/US14/66712

§ 371 Date: May 12, 2016

Related U.S. Application Data
(60) Provisional application No. 61/906,855, filed on Nov. 20, 2013.

(51) Int. Cl. C07K 14/54; C12N 15/113; G01N 33/68; C07K 16/24

(52) U.S. Cl. CPC C07K 14/54; C07K 16/244; C12N 15/1136; G01N 33/6869; C07K 2317/76; C12N 2310/14; C12N 2320/30; A61K 38/00

(57) ABSTRACT

Compositions and methods involving IL40, a novel cytokine produced by activated B cells, are provided. The compositions include: a) anti-IL40 antibodies, IL40 peptides and IL40 proteins; b) nucleic acids encoding IL40 gene and cDNA sequences; and c) pharmaceutical compositions thereof. The methods include treatments, diagnostics and isolation technologies.

### CROSS-REFERENCE TO RELATED APPLICATIONS

This application claims the benefit of U.S. Provisional Patent Application No. 61/906,855, filed on Nov. 20, 2013, which is incorporated by reference herein.

### STATEMENT REGARDING FEDERALLY SPONSORED RESEARCH OR DEVELOPMENT

This invention was made with Government support under Grant No. AI096278 from the National Institutes of Health. The Government has certain rights in this invention.

### BACKGROUND

1. Field of the Invention

The invention relates to compositions and methods involving a new cytokine called IL40.

2. Related Art

Cytokines are small secreted proteins that regulate the immune system. They are very important mediators that regulate immune responses, including the class and magnitude of the response. Examples include the interleukins, chemokines, tumor necrosis factor superfamily, and interferons. Many of these are currently becoming explored as immunotherapeutics. They are involved in autoimmune diseases, cancer, and other ailments.

### SUMMARY

Embodiments of the invention involve the identification of a novel cytokine herein called Interleukin 40 (IL40 or IL-40). The molecule is a small secreted protein produced by activated B cells. Therefore, it is a biomarker of activated B cells. It is also upregulated in certain diseases, like systemic lupus erythematosus. Because B cells are linked to lymphomas and autoimmune diseases, it is expected that IL40 plays a role in the pathology of these diseases and will be a diagnostic or prognostic biomarker. It will also impact the development of lymphomas, either by influencing the development of these cancers, or by rendering them resistant to apoptosis, enhancing their growth, or favoring their differentiation. In autoimmunity, IL40 produced by pathogenic B cells should affect other cells and favor the inflammatory responses associated with these conditions. Methods to use IL40 to identify its receptor are also contemplated. The identification of its specific receptor is important because the availability of the ligand and its receptor can be leveraged to identify agonists and antagonists of this interaction that can be used in the indications described above as well.

In one aspect, an antibody against the C17orf99 polypeptide gene product (IL40) is provided. The anti-IL40 antibody can be:

a) an IgG, IgM, IgA, IgD or IgE;

b) a monoclonal antibody;

c) an Fab′, Fab, F(ab′)₂, single domain antibody (sdAb), Fv, or scFv (single chain Fv);

d) a labeled antibody;

e) a neutralizing antibody; or

f) any combination of a)-e).

In another aspect, a method of using the anti-IL40 antibody is provided. In the method, the anti-IL40 antibody can be used, e.g.:

a) In a method of detecting IL40 in a sample. The method includes immunodetecting IL40 using the antibody as a detection agent for detecting IL40 in an immunodetection method. In some embodiments, the immunodetection method is enzyme-linked immunosorbent assay (ELISA), histology, fluorescence-activated cell sorting, radioimmunoassay (RIA), immunoradiometric assay, immunohistochemistry, fluoroimmunoassay, chemiluminescent assay, bioluminescent assay, Western blotting, or dot blotting. In an ELISA assay, two different antibodies recognizing two different epitopes of a given protein can be used to detect the protein through the detection of a substrate linked to one of the antibodies in a colorimetric assay. In Histology, a labeled antibody can be used to detect a protein in a tissue sample either in fresh frozen tissue or in formalin-fixed, paraffin embedded samples. In fluorescence-activated cell sorting, a fluorochrome-labeled antibody can be used to detect cells that express a particular protein. In the case of a secreted protein there are techniques available that allow the intracellular staining of said proteins by procedures known to those skilled in the art. In a radioimmunoassay a radioactively labeled protein can be used to measure the amount of protein present in a given sample by measuring the amount of radioactivity present in a competition assay (for example, by using a specific antibody). Variations of these assays involve the use of antibody/labeling compounds to measure the amount of a particular protein in a given sample through competition assays that depend on the affinity/avidity of the specific antibody. In a Western blot, a given protein can be detected by the use of a specific antibody following a gel transfer, a method that also allows the technician to know the molecular weight of the protein detected.

b) In a method of treating a disease involving IL40 in a subject in need thereof. The method includes administering to the subject a therapeutically effective amount of the anti-IL40 antibody to neutralize IL40. In some embodiments, the disease is an autoimmune disease or lymphoma.

c) In a method of detecting IL40 in a sample. The method includes immunodetecting IL40 using the anti-IL40 antibody as a detection agent in a diagnostic or theranostic method for a disease involving IL40. There are many methods available based on the use of antibodies to detect the presence of a soluble protein in a physiological fluid. Among the most common ones are enzyme-linked immunoassays where two different antibodies recognizing different epitopes of IL40 are used. One of them is used as a capture antibody in a plate where the physiological fluid is placed. This antibody is stuck to the plate, and “captures” IL40 present in the fluid. The second antibody is linked to an enzyme. Finally, a substrate is used that is processed by the enzyme and typically results in the development of a given color that can be detected in specialized ELISA readers. Other methods include radioimmunoassays that use radioactivity instead of enzymatic substrates to measure the amount of IL40 present in a given fluid. The fluids can be obtained from patients with different diseases. Typically, activated B cells have been found to play a role in the pathogenesis of various cancers (lymphomas, leukemias) or inflammatory or autoimmune diseases (rheumatoid arthritis, systemic lupus erythematosus, Sjogren's syndrome, ankylosing spondilytis, psoriasis, others). In some embodiments, the disease is lymphoma, leukemia, immunodeficiency or an autoimmune disease. In particular embodiments, the autoimmune disease is systemic lupus erythematosus, rheumatoid arthritis, or psoriasis, and the immunodeficiency is IgA deficiency syndrome.

d) In a method of purifying or isolating a subset of cells that express IL40. The method includes using the anti-IL40 antibody as a purification/isolating agent to purify or isolate the subset of cells. Antibodies can be purified from hybridoma cultures. Typically, the supernatants are first filtered through a 0.45 mm filter to remove cell debris. A preferred method involves protein A/G chromatography. Applying the filtered hybridoma cultures to the A/G protein column will result in the antibody molecules binding to the A/G protein, in a bond that can be subsequently broken by changing the pH and eluting the purified antibody from the column. The purified antibody can then be labeled with various fluorochromes and then used to stain cell suspensions that can be analyzed in a fluorescence-activated cell sorter. This procedure may result in the identification of cell subsets that express the antigen recognized by the antibody. In some embodiments, the subset is purified or isolated by fluorescence-activated cell sorting (FACS) to select the cell subsets.

In a further aspect, a cell producing the anti-IL40 antibody is provided, where the cell is a hybridoma, a recombinant bacterial cell, a recombinant yeast cell, or a recombinant mammalian cell. The cell can produce any of the anti-IL40 antibodies described herein. Also, an organ, tissue or animal comprising the cell is provided.

In another aspect, a peptide or isolated protein comprising a part of or the entire amino acid sequence of IL40 or mature form of IL40 is provided. The IL40 peptide or protein can be:

a) a sequence variant, polymorphism, or species counterpart of IL40;

b) a substitutional, insertional, or deletion variant of IL40;

c) a non-sequence derivative of IL40, selected from the group consisting of glycosylation modified IL40, chemically modified IL40, and a conjugate of IL40;

d) a functionally active variant of IL40;

e) a functionally active segment of IL40, a conserved region of IL40, or a non-conserved region of IL40;

f) a fusion protein of IL40; or

g) any combination of a)-f).

In some embodiments, the functional variant is an agonist or antagonist of IL40, and the fusion protein is a covalent or noncovalent construct, or a labeled construct.

IL40 should bind to its specific receptor which will be present in certain populations of lymphocytes and leukocytes in general. To identify the receptor, methods can be utilized as the described herein where IL40 is labeled with either a label (such as FLAG or HIS-tag) or radioactivity. If labeled with an amino acid based label (such as FLAG or HIS-tag) the successful binding of IL40 to its receptor can be detected by using a secondary anti FLAG or anti-HIS antibody labeled with a fluorochrome and detected in a fluorescence-activated cell sorter (FACS). If labeled with radioactivity, the binding can be monitored by measuring the radioactive counts bound to a cell expressing the receptor. The biological activity of IL40 can be monitored by measuring the expression of genes whose expression is modulated by IL40 in various leukocyte populations (see, for example those listed in Table 5). Leukocytes (for example splenocytes) can be cultured in vitro in the presence of IL40 for 6 hours before mRNA is prepared from the cells and used to measure the expression of these genes by real-time PCR. In vivo, IL40 is necessary for optimal production of IgA. Therefore, the activity of IL40 antagonists can be monitored in vivo by administering them to a mouse and measuring at various time intervals thereafter IgA levels in serum or plasma. Conversely, the activity of IL40 agonists can be measured in vivo by administering them to an IL40−/− mouse and measuring at various time intervals IgA levels in serum or plasma. Successful IL40 agonists should be able to correct the IgA defect induced by the IL40−/− mutation, and therefore IgA levels should rise to those of a normal mouse.

An IL40 fusion protein can also be used to monitor IL40 activity in vivo or in vitro, or to alter the pharmacokinetics of native IL40 in vivo. Examples of fusion proteins include but are not limited to those linked to immunoglobulin heavy chain such that fusion would result in an IL40-Fc fusion protein. This fusion protein could be more stable in vivo, or it may exhibit desirable binding characteristics by being able to bind to Fc receptors present in many leukocyte populations, which may result in preferential localization of the fusion protein to lymphoid tissues. Alternatively, IL40 could be used to make a fusion protein with other cytokines or chemokines that preferentially bind B cells. For example, IL40 could be fused to parts of the interleukin 4 (IL4) gene that encode those parts of the IL4 cytokine that bind the IL4 receptor, which is present in subsets of both B and T cells. Alternatively, IL40 could be fused to CXCL13, a chemokine that binds CXCR5, a receptor also preferentially expressed in B cells. These fusion proteins may exhibit desirable biological properties that could enhance or alter the biological responses of B lymphocytes, or their homing patterns within the human body.

IL40 can be labeled with radioactivity (amino acids or atoms) or by adding a few amino acids to its sequence. Two common labels that have been used include HIS-tag and FLAG. The latter have the advantage that there are readily available commercial monoclonal antibodies that recognize their epitopes and therefore can be used to detect the labeled IL40 when the labels are attached to it.

In a further aspect, a method of using the IL40 peptide or protein is provided. In the method, the peptide or protein can be used, e.g.:

a) In a method of inducing an immune cell. The method includes using the peptide or protein as an active agent to induce the immune cell to produce synaptogyrin 2 and/or other IL40-induced proteins produced by B cells, or to induce differentiation or maturation of the immune cell. Cells can be incubated with IL40 (at various concentrations) for 24 h in vitro in tissue culture medium, typically using RPMI 1640 or DMEM or similar supplemented with fetal calf serum, glutamine and -mercaptoethanol.

b) In a method of treating a disease involving IL40 in a subject in need thereof. The method includes administering to the subject a therapeutically effective amount of the peptide or protein, where the peptide or protein is an IL40 antagonist. In some embodiments, the disease is autoimmune disease or lymphoma.

c) In a method of treating a disease involving IL40 in a subject in need thereof. The method includes administering to the subject a therapeutically effective amount of IL40, or the peptide or protein, where the peptide or protein is an IL40 agonist. In some embodiments, the disease is IgA deficiency syndrome, Hodgkin or non Hodgkin Lymphomas, diffuse large cell lymphoma, mycosis fungoides, mantle cell lymphoma, multiple myeloma, or another lymphoma or leukemia; rheumatoid arthritis, systemic lupus erythematosus, Sjogren's syndrome, Hashimoto thyroiditis, scleroderma, Graves' disease, Crohn's disease, ulcerative colitis, primary biliary cirrhosis, autoimmune hepatitis, multiple sclerosis, psoriasis, atopic dermatitis, idiopathic pulmonary fibrosis, hypersensitivity pneumonitis, non-specific interstitial pneumonia, or another autoimmune disease.

d) In a method of diagnosing a disease involving IL40. The method includes using the peptide or protein as a target or sample control in a diagnostic/theranostic method. There are many methods available based on the use of antibodies to detect the presence of a soluble protein in a physiological fluid, including saliva, serum, plasma, semen, bronchoalveolar lavage fluid, urine, tears, lymph, sweat, bile, cerebrospinal fluid, and the like. Among the most common methods are enzyme-linked (ELISA) immunoassays where two different antibodies recognizing different epitopes of IL40 are used. One of them is used as a capture antibody in a plate where the physiological fluid is placed. This antibody is stuck to the plate, and “captures” IL40 present in the fluid. The second antibody is linked to an enzyme. Finally, a substrate is used that is processed by the enzyme and typically results in the development of a given color that can be detected in specialized ELISA readers. Other methods include radioimmunoassays that use radioactivity instead of enzymatic substrates to measure the amount of IL40 present in a given fluid. The fluids can be obtained from patients with different diseases. Typically, activated B cells have been found to play a role in the pathogenesis of various cancers or inflammatory or autoimmune diseases. In some embodiments, the disease is lymphoma, an autoimmune disease, systemic lupus erythematosus, rheumatoid arthritis, or psoriasis.

e) In a method of identifying a receptor for IL40. The method includes using the peptide or protein as a ligand for binding to the IL40 receptor. The IL40 receptor can be identified by using labeled IL40 that can be used to bind to its receptor. The ligand/receptor complex can then be immunoprecipitated using an anti-IL40 or anti-label antibody. Examples of such labels include His-Tag, Flag-tag, and the like. IL40 can also be radiolabeled to first detect via radioimmunoassay cells that express the receptor. Different cells are incubated with radiolabeled IL40, and following incubation the cells are washed or passed through gradients that separate by viscosity and centrifugation free versus bound radiolabeled IL40. Cells that retain radioactivity should express the specific IL40 receptor.

In a further aspect, a cell producing the IL40 peptide or protein is provided, where the cell is a recombinant bacterial cell, a recombinant yeast cell, or a recombinant mammalian cell. The cell can produce any of the IL40 peptides or proteins described herein. Also, an organ, tissue or animal comprising the cell is provided.

In another aspect, a nucleic acid comprising a part of or the entire nucleotide sequence of an IL40 gene or IL40 cDNA is provided, including sequences lacking one or more introns found in a native gene, or incorporating an unnatural nucleotide. The nucleic acid can be one that:

a) comprises a part of or the entire nucleotide sequence of an IL40 gene or IL40 cDNA, wherein the nucleic acid encodes an IL40 peptide or protein described herein;

b) is conjugated to another nucleotide sequence, to a label (for example, HIS-tag or FLAG), or to a chemical derivative such as a vinyl sulfone derivatized dye, fluorophore, or other tags (such as biotin) which are commonly used in other techniques such as proteomics;

c) is a primer, a probe, an antisense molecule, or an oligonucleotide based on the IL40 gene or IL40 cDNA sequence;

d) is a recombinant construct attached to a heterologous nucleic acid sequence; or

e) any combination of a)-d).

In some embodiments, the heterologous nucleic acid sequence can be a promoter, an enhancer, a vector, or an expression vector.

In a further aspect, a method of using the IL40 sequence-containing nucleic acid is provided. In the method, the nucleic acid can be used, e.g.:

a) In a method of treating a disease involving IL40 in a subject in need thereof. The method includes administering to the subject a therapeutically effective amount of the nucleic acid, where the nucleic acid decreases IL40 expression. In some embodiments, the disease is an autoimmune disease or lymphoma.

b) In a method of treating a disease involving IL40 in a subject in need thereof. The method includes administering to the subject a therapeutically effective amount of the nucleic acid, wherein the nucleic acid increases IL40 expression. In some embodiments, the disease is IgA deficiency syndrome, Hodgkin or non Hodgkin Lymphomas, diffuse large cell lymphoma, mycosis fungoides, mantle cell lymphoma, or another lymphoma or leukemia; rheumatoid arthritis, systemic lupus erythematosus, Sjogren's syndrome, Hashimoto thyroiditis, scleroderma, Graves' disease, Crohn's disease, ulcerative colitis, primary biliary cirrhosis, autoimmune hepatitis, multiple sclerosis, psoriasis, atopic dermatitis, idiopathic pulmonary fibrosis, hypersensitivity pneumonitis, non-specific interstitial pneumonia, or another autoimmune disease. Also, in some embodiments, the nucleic acid can be an RNAi molecule.

c) In a method of diagnosing a disease involving IL40. The method includes using the nucleic acid as a probe in a diagnostic/theranostic method for the disease. In some embodiments, the disease is lymphoma, autoimmune disease, systemic lupus erythematosus, rheumatoid arthritis, or psoriasis.

In a further aspect, a cell comprising a recombinant form of the IL40 sequence-containing nucleic acid is provided, where the cell is a recombinant bacterial cell, a recombinant yeast cell, or a recombinant mammalian cell. The cell can comprise any of the IL40 sequence-containing nucleic acids described herein. Also, an organ, tissue or animal comprising the cell is provided

In another aspect, a method of selecting a subset of cells expressing IL40 is provided. The method includes adding a molecule that binds IL40 to a cell population comprising cells expressing IL40, and selecting cells labeled with the IL40 binding molecule to provide a population of selected cells. Cells expressing IL40 include B-cells, possibly other leukocytes, and bone marrow and fetal liver cells. The cell types that may express IL40 is these organs may include epithelial, endothelial, fibroblasts, other stromal cells, or hematopoietic precursors of various cell types or levels of commitment to a certain lineage. In some embodiments: a) the cells expressing IL40 can be mouse, rat or human cells; b) the IL40 binding molecule can be an anti-IL40 antibody or an IL40 receptor; c) the selected cells can be selected from blood, body fluids, cell suspensions or patient samples; d) the selected cells can be research tools for studying IL40 expressing cells; e) also, when the selected cells are blood cells, the selected cells can be: i) a source of mRNA for immunoglobulins produced by the selected cells; or ii) a source of new methods for producing fully humanized antibodies; or f) any combination of a)-e).

In a further embodiment of the method, a method of treating a disease involving IL40 in a subject in need thereof is provided. The method includes administering to the subject a therapeutically effective amount of the selected cells. In some embodiments, the disease is IgA deficiency syndrome, Hodgkin or non Hodgkin Lymphomas, diffuse large cell lymphoma, mycosis fungoides, mantle cell lymphoma, or another lymphoma or leukemia; rheumatoid arthritis, systemic lupus erythematosus, Sjogren's syndrome, Hashimoto thyroiditis, scleroderma, Graves' disease, Crohn's disease, ulcerative colitis, primary biliary cirrhosis, autoimmune hepatitis, multiple sclerosis, psoriasis, atopic dermatitis, idiopathic pulmonary fibrosis, hypersensitivity pneumonitis, non-specific interstitial pneumonia, or another autoimmune disease.

In another aspect, a method of detecting activated B-cells in a subject is provided. The method includes measuring the level of IL40 in the subject, wherein an increased level of IL40 over control is indicative of activated B-cells. In the method, e.g.:

a) the IL40 level can be measured by an immunodetection technique;

b) the method can further include measuring another biomarker such as, but not limited to, interleukin 6, interkeukin 10, and certain immunoglobulins;

c) the method can diagnose autoimmunity or lymphoma in the subject in need of such diagnosis, where the increased level is indicative of lymphoma or autoimmunity;

d) the increased level of IL40 can define an IL40-producing subtype of lymphoma or autoimmune disease; or

e) any combination of a)-d).

In some embodiments, the immunodetection technique is ELISA, histology, fluorescence-activated cell sorting, radioimmunoassay (RIA), immunoradiometric assay, immunohistochemistry, fluoroimmunoassay, chemiluminescent assay, bioluminescent assay, Western blotting or dot blotting.

In another aspect, a method of treating lymphoma or autoimmune disease in a subject in need thereof is provided. The method includes administering a therapeutically effective amount of an anti-IL40 antibody, or an IL40 sequence-containing nucleic acid, to the subject, or to a tumor, tissue or cell of the subject. In some embodiments, the antibody can be a neutralizing anti-IL40 antibody, and the nucleic acid can be an antisense RNA. In some embodiments, the antisense RNA is an RNAi molecule.

In another aspect, a method of identifying IL40-producing cells in a subject is provided. The method includes using an anti-IL40 antibody, or an IL40 sequence-containing nucleic acid, as a probe to identify the IL40-producing cells, for example, in immunohistochemistry or in situ hybridization. An IL-40 antibody can be used to detect IL40 producing cells by flow cytometry or can be used to perform immunohistochemistry, while an IL40-sequence-containing nucleic acid probe can be used to run a Northern blot of mRNA obtained from cells that produce IL-40. The IL40 sequence can be used to design primers to perform real-time polymerase chain reaction (PCR) on mRNA obtained from IL40-producing cells. The IL40 sequence probe can be used to identify cells producing IL40 by in-situ hybridization.

In a further aspect, a method of identifying an IL40 receptor is provided. In some embodiments, the method includes:

a) Labeling an IL40-responsive cell with IL40, a labeled IL40, a His-tagged IL40, a biotin-labeled IL40, or a combination thereof, and isolating, purifying and/or separating the labeled cell. The cells labeled with these labels can be separated, for example, by using a fluorochrome-labeled specific antibody against HIS-tag and running the sample through a fluorescence-activated cell sorter or, in the case of biotin-labeled IL40, by using fluorochrome-labeled streptavidin in a similar method; or

b) Labeling an IL40-responsive cell with IL40, a labeled IL40, a His-tagged IL40, a biotin-labeled IL40, or a combination thereof, and isolating, purifying and/or separating the labeled cell, wherein the cell is a eukaryotic or bacterial cell expressing the IL40 receptor; or

c) Identifying a protein that binds to IL40 in a yeast two-hybrid system; or

d) Immunoprecipitating an IL40-binding protein from a membrane preparation from cells expressing the IL40 receptor. For example, cells that express the IL40 receptor can be disrupted using tiny glass, ceramic or steel beads mixed with a cell sample in aqueous media. The mix is subjected to high level agitation by stirring and shaking Beads collide with the cells and break them to release intercellular components. Mechanical shear (vortexing) is moderate during homogenization and results in excellent membrane or subcellular preparations that can be separated by centrifugation. Immunoprecipitation of protein complexes (as in labeled IL40 bound to its receptor) can be achieved by using an antibody against the label (which could be HIS-tag). Analysis and sequencing of the immunoprecipitate should lead to the identification of proteins that are present in this complex.

In another aspect, a method of using IL40 or a functionally active variant thereof is provided. The method includes exposing immune cells to IL40 or to the functionally active variant so as to:

a) promote growth and differentiation of B cells in vitro or in vivo;

b) increase growth of a hybridoma culture;

c) increase antibody production by a hybridoma culture; or

d) determine the species origin of a tissue or cell by using IL40 from different mammalian species, such as human, dog, mouse, cat, cow, horse, pig, goat, or sheep.

For example, B lymphocytes can be grown and differentiated in vitro by culture in regular tissue culture medium supplemented with various cytokines (IL4, IL6) and antibodies that stimulate the B cell receptor (anti-immunoglobulin), or molecules that bind the CD40 receptor (CD40 ligand or antibodies against CD40 receptor). These conditions lead to the growth and/or differentiation of B lymphocytes. Other cytokines known to favor differentiation of B cells include IFNγ, TGFβ, IL5, IL13, and CXCL13. B cell hybridomas that result from the fusion of a normal B cell with a B cell myeloma or other tumor cell can be cultured in vitro in selective media (to favor the growth of the hybridoma) supplemented with cytokines that favor growth of the hybridoma (IL6).

In any of the foregoing compositions or methods involving autoimmunity, autoimmune disease or lymphoma,

the autoimmune disease can be systemic lupus erythematosus, rheumatid arthritis, psoriasis, Graves' disease, autoimmune hepatitis, primary biliary cirrhosis, Hashimoto's thyroiditis, or Sjögren's syndrome, and

the lymphoma can be Hodgkin's and non-Hodgkin's lymphoma, Mantle cell lymphoma, Diffuse large B cell lymphoma, Follicular lymphoma, Chronic lymphocytic leukemia, Acute lymphocytic leukemia, MALT lymphoma, Burkitt's lymphoma, Mycosis fungoides, or multiple myeloma.

In any of the foregoing compositions or methods involving treating or treatment, the antibody, peptide, protein or nucleic acid can be delivered locally or systemically.

In any of the foregoing compositions or methods involving diagnostic, diagnosis, diagnose or diagnostic/theranostic methods, the methods can be practiced on a sample, such as serum, blood, a body fluid, a tumor, a tissue or a cell, including a biopsy or histology sample.

In any of the foregoing compositions or methods involving antibody, peptide, protein or nucleic acid molecules, the molecules can be in a pharmaceutical formulation that:
a) comprises a pharmaceutically acceptable carrier, excipient, or a combination thereof;
b) is used as a sterile formulation;
c) comprises another therapeutic agent for treating autoimmune disease or lymphoma, such as but not limited to, anti-TNFa antibodies (Remicade, Humira); anti-BAFF (Benlysta); anti-CD20 (Rituximab); and anti-CD30 (Adcetris);
d) is in a slow or sustained release formulation, e.g., emulsions, micelles, etc.;
e) is in a targeted administration form, e.g., liposome, inclusion complexes, carriers; or
f) any combination of a)-e).

### BRIEF DESCRIPTION OF THE DRAWINGS

For a more complete understanding of the present invention, reference is now made to the following descriptions taken in conjunction with the accompanying drawings, in which:

FIG. 1 is a panel showing that C17orf99 is a novel cytokine expressed in the fetal liver, bone marrow, and activated B cells. 1A) C17orf99 expression in normal human tissues and immune cells. Data from the BIGE (Body Index of Gene Expression) database (human microarray data using the Affymetrix genearray (U133 2.0)) showing the expression profile of C17orf99 mRNA. X-axis is organized by organ systems: CNS (central nervous system), Gut (gastrointestinal), Struct (structural), Vasc (vasculature), Resp (respiratory), Endo (endocrine), Ur (urinary), Rep (reproductive), Imm_T (immune tissue), Imm_C (immune cells), and Dev (developmental). Y axis represents hybridization intensity of the probe set corresponding to C17orf99 (236981_at). 1B) C17orf99 human amino acid sequence (SEQ ID NO. 1) showing the signal peptide. 1C) Western blot of supernatant from pTT5V5H8-C17orf99 transfected 293 HEK cells. 293 HEK cells were transfected with either pTT5V5H8-C17orf99 or empty vector. The supernatant was collected and purified using a GE HisTrap purification column and western blotting was performed using an anti-His mAb followed by anti-rabbit HRP secondary antibody. 1D) RT-PCR confirmation of C17orf99 expression in human fetal liver and bone marrow. 1E) Clustal Omega analyses of the amino acid sequence of C17orf99 in 10 mammalian species. C17orf99 homologs were restricted to placental mammals (eutheria), marsupials and monotremes.

FIG. 2 is a panel of graphs showing that IL40 expression is similar in mice and humans. 2A) qRT-PCR analysis of human samples from commercially available RNAs. 2B) qRT-PCR analysis of mouse tissue obtained from commercially available RNAs.

FIG. 3 is an amino acid sequence comparison between human (SEQ ID NO. 2) and mouse (SEQ ID NO. 3) IL40 proteins. The analysis was performed using the BLAST program. A consensus sequence (SEQ ID NO. 4) is also shown.

FIG. 4 is a panel of graphs showing that IL40 is expressed in human and mouse activated B cells. 4A) qRT-PCR analysis for IL40 and TSPAN33 of human resting and activated B cells for 24 h with anti-CD40 mAb+IL4. 4B) qRT-PCR analysis of IL40 and Tspan33 of mouse splenocytes under resting or stimulated conditions for 24 h with 1 ug/mL LPS+IL-4. 4C) qRT-PCR analysis of FACS purified CD19+ B cells from a spleen of a C57BL/6 mouse for IL40 and Tspan33 stimulated with CD40L+IL-4, for 8, 24, 72, and 96 h. 4D) qRT-PCR analysis of splenocytes stimulated with 2 ng/mL of IL-4, IL-13, IFNγ, or TGFβ, for 3 days. 4E) qRT-PCR analysis of IL40 and Tspan33 expression stimulated with combinations anti-CD40 and cytokines

FIG. 5 is a panel of graphs of qRT-PCR analysis of IL40 in human and B cell lines. 5A) Human 2E2 B cells and human Jurkat T cells were measured for IL40 expression under resting and stimulating conditions. 5B) Murine A20-2J mouse cells were measured for Il40 expression under resting and stimulated conditions.

FIG. 6 is a panel showing the results of FACS purification of CD19+ B cells from mouse splenocytes. 6A) CD19+ cells in mouse splenocytes before sorting. 6B) CD19+ cells in mouse splenocytes after sorting.

FIG. 7 is a graph showing that IL40 transcription is induced by LPS+TGFβ. qRT-PCR analysis of mouse splenocytes stimulated with IL-4, LPS, or combinations of LPS and IL-4, IL-13, IFNγ, or TGFβ.

FIG. 8 is a panel showing that IL40 deficient mice have an altered B cell phenotype. 8A) Target construct used to generate IL40−/− mice. 8B) Photograph of a spleen from a WT (left) and IL40−/− (right) mouse. 8C) Ratios of CD19+ (B cells) vs. CD3e+ (T cells) in the spleens of WT and IL40−/− mice, measured by flow cytometry. 8D) Measurements of spleen size (left), weight (middle), and total lymphocyte numbers (right), in WT and IL40 deficient mice. (n=5 per group). 8E) Graph of Ratios obtained from C (left) and total numbers of T and B cells in WT and IL40 deficient mice (n=5). 8F) Serum levels of IgG1 (left) and IgA (right) in wildtype and IL40−/− mice measured by sandwich ELISA. (n=5). The figures depict representatives from at least 3 independent experiments.

FIG. 9 is a photograph indicating PCR confirmation of genotyping from WT and IL40−/− colonies. Prominent bands at 250 bp (IL40 genomic DNA) and 200 bp (Neomycin construct) are seen. DNA obtained from WT mice only contain the IL40 genomic DNA, while mice with the deletion carry only the neomycin construct. Heterozygotes contain both the IL40 genomic DNA and the neomycin construct.

FIG. 10 is a graph showing that IL40−/− mice do not have defects in weight gain. Mice from WT and IL40−/− mice were measured from 3-6 weeks for weight (g). n=5.

FIG. 11 is a panel showing that IL40−/− mice do not have defects in T cell developments in the thymus. 11A) Thymocytes were measured, gated on CD3e expression, then measured for CD4 vs. CD8 expression using flow cytometry. 11B) Percentage of positive cells. The figure depicts experiments obtained from 2 independent experiments, n=3.

FIG. 12 is a panel showing that IL40−/− mice do not have defects in IgA production in B1 cells from peritoneal cavity of B2 cells from the resting spleen. Spleen or peritoneal cavity cells were gated on subpopulations of CD5 or B220+ cells to identify/gate populations of B1a, B1b, or B2 cells and then their expression of IgA was measured by flow cytometry, (n=3).

FIG. 13 is a panel showing that IL40 deficient mice do not have defects in Pro, Pre, or Immature B cell populations. 13A) Bone marrow exudate cells were gated on B220+CD19+ cells and measured for IgM vs CD43 expression. 13B) Percentage of cells measured for 3 separate mice. 13C) Total cell numbers measured for 3 separate mice. Representative experiment obtained from 2 independent experiments.

FIG. 14 is a panel showing that splenocyte populations of B cells are normal in resting IL40 deficient mice. 14A) Mice were gated on B220+ cells, then stained for IgM vs. IgD expression. 14B) B220+ cells were then measured for IgM vs. IgA expression. 14C) B220+ cells were stained with 7AAD to measure cell viability. N=3 mice per group.

FIG. 15 is a panel showing that IL40−/− mice have a defect in IgA producing cells in the Peyer's Patches. 15A) Measurement of total germinal center (B220+PNA+) cells in the Peyer's Patches, n=5. 15B) Measurement of IgA secreting plasma cells, gated on B220+PNAhi lymphocytes, n=5. 15C) Measurement of total IgA switched cells, B22010-hiPNA+ lymphocytes, n=5. 15D) Measurements of total IgA in fecal pellets, n=10. 15E) IL40 transcription is upregulated in the mammary glands of lactacting females. qRT-PCR analysis obtained from mammary glands of a virgin, pregnant, and lactacting 1 wk and 3 wk mouse. 15A-D are representative of 3 independent experiments, with at least 3 mice per group.

FIG. 16 is a panel showing that IL40−/− mice do not have a B cell intrinsic in the ability to undergo CSR, during in vitro induction assays. CSR induction of mouse splenocytes stimulated for 4 days, stimulated with: 16A) LPS+anti-BCR+TGFβ (IgA switching), 16B) LPS+IL-4 (IgG1 switching), 16C) LPS+IFNγ (IgG3 switching), measured by flow cytometry. 16D) Stimulation of IgG1 plasma cells with LPS+IL-4. Representative data obtained from at least 2 independent experiments, with at least 3 groups per mice.

FIG. 17 is a graph showing that human synaptogyrin 2 is expressed in activated B cells. Expression profile of human tissues obtained from the BIGE.

FIG. 18 is a panel showing that IL40 affects only B cells, not T cells. Microarray analysis of “B cell” and “T cell” genes from WT and IL40−/− mouse splenocytes, under resting (left), and activated (right) conditions.

FIG. 19 is a panel showing that IL40 is elevated in MRLFas^{lpr/lpr} mice. qRT-PCR of IL40 and Tspan33 expression of total splenocytes taken from MRL/faslpr/lpr mice normalized to CD19 expression. Mice ages 9 weeks old (no detectable pathology), 24 weeks old (lymphadenopathy with or without mild ear lesions) and 36 weeks old (lymphadenopathy with ear and face lesions) were compared for Tspan33 expression, n=5.

FIG. 20 is a panel showing that IL40 binds to B cells but not T cells.

FIG. 21 shows the nucleotide sequence (SEQ ID NO. 5) of the coding region of a cDNA encoding the human IL40 protein.

### DETAILED DESCRIPTION

Antibodies, peptides, proteins and nucleic acids related to the gene product of the gene C17orf99 are included in various embodiments. The nucleotide sequences of the C17orf99 gene and C17orf99 cDNA from various species, and the amino acid sequences of the C17orf99 gene product from various species, have the following accession numbers (all incorporated by reference herein): human C170RF99: NM001163075; mouse C170RF99: NM 029964 (see National Center for Biotechnology Information, on the world wide web at ncbi.nlm.nih.gov). As used herein, the C17orf99 gene product is also referred to as interleukin-40 (IL40 or IL-40).

An antibody is any immunologic binding agent such as IgG, IgM, IgA, IgD and IgE. An antibody can also be any antibody-like molecule that has an antigen binding region, and includes antibody fragments such as Fab′, Fab, F(ab′)₂, single domain antibodies (DABs), Fv, scFv (single chain Fv), and the like. Techniques for preparing and using various antibody-based constructs and fragments are well known in the art. Means for preparing and characterizing antibodies are also well known in the art (See, for example, Harlow and Lane, “Antibodies: A Laboratory Manual,” Cold Spring Harbor Laboratory, 1988). Monoclonal antibodies (mAbs) are recognized to have certain advantages, e.g., reproducibility and large-scale production. Thus, monoclonal antibodies of the human, murine, monkey, rat, hamster, rabbit and even chicken origin, are contemplated.

Polyclonal antibodies against IL40 can be prepared in a wide range of animal species. Typically, the animal used for production of antisera is a rabbit, a mouse, a rat, a hamster, a guinea pig or a goat. To increase immunogenicity, use of adjuvants and conjugation to a carrier protein such as, but not limited to, keyhole limpet hemocyanin or bovine serum albumin are well known procedures.

A monoclonal antibody can be readily prepared through use of well-known techniques, such as those exemplified in U.S. Pat. No. 4,196,265, incorporated herein by reference (40-44). Typically, this technique involves immunizing a suitable animal with a selected immunogen composition, e.g., a purified or partially purified protein, peptide or domain. The immunizing composition is administered in a manner effective to stimulate antibody producing cells (45-47). Hybridomas secreting monoclonal antibodies can be isolated.

A polyclonal or monoclonal antibody can be further purified, if desired, using filtration, centrifugation and various chromatographic methods such as HPLC or affinity chromatography (47).

Humanized monoclonal antibodies are antibodies of animal origin that have been modified using genetic engineering techniques to replace constant region and/or variable region framework sequences with human sequences, while retaining the original antigen specificity. Such antibodies are commonly derived from rodent antibodies with specificity against human antigens. Such antibodies are generally useful for in vivo therapeutic applications. This strategy reduces the host response to the foreign antibody and allows selection of the human effector functions. Thus, humanized antibodies against IL40 are included in some embodiments of the invention, as are chimeric antibodies from mouse, rat, or other species, bearing human constant and/or variable region domains, bispecific antibodies, recombinant and engineered antibodies and fragments thereof. The techniques for producing humanized immunoglobulins are well known to those of skill in the art (44, 47-51). For example U.S. Pat. No. 5,693,762 discloses methods for producing, and compositions of, humanized immunoglobulins having one or more complementarity determining regions (CDR's). When combined into an intact antibody, the humanized immunoglobulins are substantially non-immunogenic in humans and retain substantially the same affinity as the donor immunoglobulin to the antigen, such as a protein or other compound containing an epitope. Examples of other teachings in this area include U.S. Pat. Nos. 6,054,297; 5,861,155; and 6,020,192, all specifically incorporated by reference. Methods for the development of antibodies that are “custom-tailored” to the patient's disease are likewise known and such custom-tailored antibodies are also contemplated.

Some embodiments of the invention include IL40 peptides or proteins. In certain embodiments, naturally occurring IL40 proteins can be substituted by IL40 variants such as substitutional, deletion and/or insertion variants.

A substitutional variant contains an exchange of one amino acid for another at one or more sites within the protein. The substitution is typically a conservative substitution involving the exchange of amino acids that are similar in shape and/or charge. A deletion variant lacks one or more residues of the native protein. An insertion mutant or variant includes the addition of one or more amino acids at a non-terminal point in the protein. Variants can have about 80% or more identity, about 85% or more identity, or about 90% or more identity, about 95% or more identity, or about 100% identity, to the naturally occurring IL40 protein sequence. A sequence comparison can be performed, for example, using Clustal Omega, MUSCLE, MView, or MAFFT sequence comparison programs. In comparing sequences, a segment of comparison between one protein and another may be about 100% of the amino acids of the length being compared, or about 95%, about 85%, or about 80% of the amino acids of the length being compared. The length of comparison may be at least about 20, 30, 40, 50, 55, 60, 65, 70, or 75 amino acids, or more. The variants may conserve particular physicochemical or functional features as the prevailing natural sequence, while other variants may have modified combinations of structural and functional features. Thus, some embodiments include functionally active IL40 variants having some or all of the functions of IL40, such as binding to the IL40 receptor or involvement in the differentiation of B cells towards IgA responses. Also, some embodiments include variants that function as IL40 agonists or IL40 antagonists. In some embodiments, the variants do not include sequences identical to naturally occurring human IL40 sequences, or naturally occurring IL40 sequences of other species. IL40 peptides and substitutional, deletion and/or insertion variants thereof are also contemplated, including functionally active IL40 peptides and IL40 peptide variants, and IL40 peptides and IL40 peptide variants that function as IL40 agonists or IL40 antagonists. In some embodiments, the peptide variants do not include amino acid sequences identical to naturally occurring amino acid sequences present in IL40 proteins of human or other species.

Certain embodiments include truncated versions of IL40 proteins, or fusions with other segments, which exhibit a function as described. A fusion protein can contain all or a portion of IL40 linked to all or a portion of a second protein. For example, the C-terminus of one protein can be linked to the N-terminus of the other protein. Alternatively, the proteins can be noncovalently linked, for example, to integrins, fibronectin receptors, or other membrane glycoproteins. The IL40 protein can contain a naturally occurring IL40 amino acid sequence, or a variant thereof.

In some embodiments, derivatives of IL40 not involving an amino acid variation, or in addition to amino acid variation, are provided. Examples of such derivatives include glycoslylation modified IL40 proteins, chemically modified IL40 proteins such as proteins modified with polyethylene glycol (pegylation), and IL40 conjugates such as ¹³¹I labeled IL40, biotin-IL40, and the like.

Some embodiments of the invention include nucleic acids encoding all or a portion of an IL40 protein, including naturally occurring IL40 proteins or variants thereof. The nucleic acid can be a DNA or an RNA molecule. The nucleic acid can be conjugated to another nucleic acid sequence, such as for expression purposes, conjugated to a label for detection purposes, or conjugated to a chemical derivative for detection purposes. For example, the nucleic acid can be conjugated to a label such as green fluorescence protein (GFP), or conjugated to a chemical derivative such as biotin.

The nucleic acid can be utilized as a primer for amplifying or synthesizing IL40 nucleotide sequences, or as a probe for identifying IL40 nucleotide sequences. In some embodiments, the nucleic acid is an oligonucleotide containing IL40 gene or IL40 cDNA sequences. In certain embodiments, the nucleic acid is an antisense molecule.

An antisense oligonucleotide is an oligomer or polymer of ribonucleic acid (RNA) or deoxyribonucleic acid (DNA) that can include naturally occurring nucleotides and/or modified or substituted oligonucleotides. In various embodiments, an antisense oligonucleotide includes a nucleotide sequence that hybridizes to an IL40 target sequence, and can include additional 5′ and/or 3′ flanking sequences, for example, for use as a primer binding site. In some embodiments, the antisense oligonucleotide can include modified oligonucleotide backbones such as, but not limited to, phosphorothioates, chiral phosphorothioates, phosphorodithioates, phosphotriesters, aminoalkylphosphotriesters, methyl and other alkyl phosphonates (e.g., 3′-alkylene phosphonates and chiral phosphonates), phosphinates, phosphoramidates (e.g., 3′-amino phosphoramidate and aminoalkylphosphoramidates), thionophosphoramidates, thionoalkylphosphonates, thionoalkyl phosphotriesters, and boranophosphates having normal 3′-5′ linkages, as well as 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. References that teach the preparation of such modified backbone oligonucleotides are provided, for example, in U.S. Pat. Nos. 4,469,863 and 5,750,666, all incorporated by reference herein. The design and synthesis of antisense oligonucleotides is well known in the art (52). Computer programs for the design of antisense oligonucleotide sequences are also available (53).

Standard methods for producing and making peptides, proteins and nucleic acids can be applied. Standard recombinant methods can be developed, including design of recombinant nucleic acids encoding constructs. See, e.g., Thompson D. A. Cell and Molecular Biology Manual 2011. Expression vectors, e.g., with promoters operably linked to coding regions, can be devised. Cells comprising the vectors are provided, including recombinant prokaryote cells and recombinant eukaryote cells such as recombinant yeast and recombinant mammalian cells. Compatible expression methodologies can also be developed.

For example, a polynucleotide that encodes an IL40 protein or protein variant can be placed under the control of a promoter that is functional in the desired host cell. An extremely wide variety of promoters is well known, and can be used in expression vectors of embodiments of the invention, depending on the particular application. Ordinarily, the promoter selected depends upon the cell in which the promoter is to be active. Other expression control sequences such as enhancers, ribosome binding sites, transcription termination sites and the like are also optionally included. Constructs that include one or more of these control sequences are termed “expression cassettes.” Accordingly, embodiments of the invention provide expression cassettes into which the nucleic acids that encode the relevant functional proteins are incorporated for high level expression in a desired prokaryotic or eukaryotic host cell (see, e.g., Ream W and Field K. G. Molecular Biology Techniques. Academic Press. 2012).

Substantially pure compositions of peptides or proteins of at least about 70%, 75%, 80%, 85%, or 90% homogeneity are included in some embodiments, with about 92%, 95%, 98%, or 99% or more homogeneity also included. The purified peptides and proteins can be used, e.g., as immunogens for antibody production, as active agents for inducing differentiation, maturation or protein expression in immune cells, or as therapeutic agents in a pharmaceutical composition.

The level of IL40 can be measured at the nucleic acid or protein level. For example, the amount of IL40 mRNA expressed in a cell can be measured, or the amount of IL40 protein present in activated B-cells can be measured. Quantitation of mRNA can be performed using methods such as, but not limited to, PCR, microarray technologies, or Northern blots (54,55). Quantitation of protein can be performed using immunodetection methods such as, but not limited to, enzyme linked immunosorbent assay (ELISA), radioimmunoassay (RIA), immunoradiometric assay, fluoroimmunoassay, chemiluminescent assay, bioluminescent assay, or Western blotting, FACS with anti-protein specific antibodies (for production by cells). The control level can be an average or mean value of IL40 levels from a control population of cells or from one or more control subjects.

In some embodiments, a diagnosis that a subject has a disease involving IL40 can be followed by a treatment such as those described herein. For example, the diagnosis can be followed by a treatment that involves administering a therapeutically effective amount of an IL40 antagonist to a subject diagnosed with autoimmunity or lymphoma, or by administering to a subject a therapeutically effective amount of an oligonucleotide containing an IL40 nucleotide sequence.

Some embodiments involve therapeutic uses of various embodiments of the invention. In these embodiments, a subject can be administered a therapeutically effective amount of an active agent, which can be an antibody, peptide, protein or nucleic acid, or any combination thereof, of various embodiments of the invention. A therapeutically effective amount is an amount that promotes or enhances the well-being of the subject with respect to the medical treatment of his/her condition. For example, extension of the subject's life by any period of time, a decrease in pain to the subject that can be attributed to the subject's condition, a decrease in the severity of the disease, an increase in the therapeutic effect of a therapeutic agent, an improvement in the prognosis of the condition or disease, a decrease in the amount or frequency of administration of a therapeutic agent, an alteration in the treatment regimen of the subject that reduces invasiveness of treatment, and a decrease in the severity or frequency of side effects from a therapeutic agent. With respect to the treatment of lymphoma or leukemia, therapeutic benefits also include a decrease or delay in the neoplastic development of the disease, decrease in hyperproliferation, reduction in tumor growth, delay of metastases, and reduction in cancer cell or tumor cell proliferation rate. The amount of active substance to be administered to the subject varies according to the weight of the subject, the mode of administration, and the indication and the severity of the disease, from which a skilled practitioner can determine a suitable dose.

In some cases, an antisense molecule containing IL40 gene or IL40 cDNA sequences can be used as a therapeutic agent to decrease expression of IL40 in a subject having a disease involving IL40. For example, the antisense molecule can be an siRNA. An siRNA is a small inhibitory RNA duplex for use in RNA interference (RNAi) methods. RNAi is a naturally occurring gene-silencing process in which double-stranded RNA is cleaved to smaller double-stranded segments (siRNA), which then associate with a protein-RNA complex (called “RISC”) leading to cleavage of target mRNA (56). In various embodiments, an siRNA can be 18-30 base pairs in size with varying degrees of complementarity to its target IL40 mRNA. In some embodiments, the siRNA can include unpaired bases at the 5′ and/or 3′ end of either or both the sense strand and antisense strand. The siRNA in some embodiments can be a duplex of two separate strands, or a single strand that forms a hairpin structure to form a duplex region. The design and synthesis of siRNAs is well known in the art (57). Computer programs for the design of siRNAs are also available (58). Other RNAi molecules include micro RNAs that are genomically encoded RNAs and that may regulate gene expression of IL40.

The subject can be a human, dog, mouse, cat or other mammal such as cow, horse, pig, goat, or sheep. In some embodiments, the subject is a subject suspected of having a disease involving IL40. In some embodiments, the subject is a subject or patient in need of treatment for a disease involving IL40.

Samples for analysis, diagnosis, and theranosis can be from a human, dog, mouse, cat or other mammal such as cow, horse, pig, goat, or sheep.

Different formulations for administration can be used (sterile, buffered, slow release, controlled release, stabilizers, ointments, etc.) depending on the optimal route of administration. See, e.g., Niazi S. K. Handbook of Pharmaceutical Manufacturing Formulations Informa Healthcare 2012. As with anti-inflammatories, agonists or antagonists of the IL40/IL40 receptor interaction can be used in combination with other established drugs to optimize therapeutic outcomes. In addition, the compound(s) can be used in combination with other therapeutics in a single formulation strategy. Phamacological variants can be used to obtain desired pharmacokinetic outcomes (secretion, half life, solubility or optimize excretion routes).

The exact dose will depend on the purpose of the treatment, and will be ascertainable by one skilled in the art using known techniques. See, e.g., Ansel, et al., Pharmaceutical Dosage Forms and Drug Delivery; Lieberman (1992) Pharmaceutical Dosage Forms (vols. 1-3), Dekker, ISBN 0824770846, 082476918X, 0824712692, 0824716981; Lloyd (1999) The Art, Science and Technology of Pharmaceutical Compounding; and Pickar (1999) Dosage Calculations. As is known in the art, adjustments for protein degradation, systemic versus localized delivery, and rate of new protease synthesis, as well as the age, body weight, general health, sex, diet, time of administration, drug interaction, and the severity of the condition may be necessary, and will be ascertainable with some experimentation by those skilled in the art.

Various pharmaceutically acceptable excipients are well known in the art. As used herein, “pharmaceutically acceptable excipient” includes a material which, when combined with an active ingredient of a composition, allows the ingredient to retain biological activity and without causing disruptive reactions with the subject's immune system. Such may include stabilizers, preservatives, salt or sugar complexes or crystals, and the like. See, e.g., Niazi S. K. Handbook of Pharmaceutical Manufacturing Formulations Informa Healthcare 2012.

Exemplary pharmaceutically carriers include sterile aqueous of non-aqueous solutions, suspensions, and emulsions. Examples include, but are not limited to, standard pharmaceutical excipients such as a phosphate buffered saline solution, water, emulsions such as oil/water emulsion, and various types of wetting agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. In other embodiments, the compositions will be incorporated into solid matrix, including slow release particles, glass beads, bandages, inserts on the eye, and topical forms. Administration routes may include the following: topical, systemic, respiratory, oral, eye, implant, vaginal, anal, suppository, devices with control release, etc.

For in vivo administration of nucleic acid compounds, the nucleic acid can be administered as a free (or “naked”) nucleic acid, or can be formulated with a delivery agent that increases delivery of the nucleic acid to a cellular target. Examples of delivery agents include, but are not limited to, liposomes, cationic lipids, PEGylated polycations, cationic block copolymers, and polyethyleneamine complexes (59).

Existing therapeutics for the indications described elsewhere in this application can be used in combination or sequentially with the agonists/antagonists of the IL40/IL40 receptor interaction to optimize therapeutic outcomes.

An IL40 gene, cDNA, nucleic acid, peptide or protein described herein can be based on human IL40 nucleotide or amino acid sequences, or based on nucleotide and amino acid sequences from another mammal such as dog, mouse, cat, cow, sheep, goat, pig or horse. Similarly, the antigen used to make an anti-IL40 antibody can be based on a human IL40 antigen, or based on an IL40 antigen from another mammal such as dog, mouse, cat, cow, horse, pig, goat, or sheep.

The present invention may be better understood by referring to the accompanying examples, which are intended for illustration purposes only and should not in any sense be construed as limiting the scope of the invention.

### EXAMPLE 1

### Introduction

The recently characterized cytokine-producing B cell subsets, namely, B regulatory cells (Breg/B10), B effector 1 (Be1), and B effector 2 (Be2) cells, provide evidence indicating that the role of B cells in inflammation and autoimmune diseases goes beyond antibody production. In particular, they point to their capacity to produce cytokines Here, a novel cytokine produced by activated B cells is described which has been named interleukin-40 (IL40). IL40 is encoded by an uncharacterized gene (C17ORF99) which is expressed in fetal liver and bone marrow in both mouse and human. It is also expressed in B cells stimulated with CD40L (or LPS). Its production is potentiated by some cytokines (IL-4, IL-13, and TGFβ, and inhibited by IFNγ. IL40 is a secreted protein of 24 kD, and is not related to other known cytokines IL40 deficient mice (Il40^{-/-}) exhibit splenomegaly, and an altered B cell phenotype, with increased CD19+ B cell numbers, and reduced numbers of IgA-producing cells in the Peyer's patches. IL40 transcripts are induced in the lactating mammary gland. Additionally, IL40 transcripts are elevated in PBMCs from patients with SLE and splenocytes from MRLFas^{lpr/lpr} mice. It is concluded that IL40 is a novel B-cell-derived cytokine with pleiotropic effects on the development and differentiation of B cells.

Cytokines are a large and diverse superfamily of pleiotropic secreted proteins, with activities that impact cellular growth, differentiation, modulation of inflammation, and hematopoiesis (1, 2). The identification of novel cytokines and their receptors was pivotal in elucidating the mechanisms through which cytokines modulate human disease. This information was critical for the diagnosis, treatment, and prevention of these diseases (3). Given their importance, there was strong interest in the search for novel cytokines, such as sequence prediction software and database searches (4). This resulted in the identification of many cytokines, most of them belonging to superfamilies that likely arose through gene duplication.

### A. Identification of IL40 as a Cytokine

The inventors have been interested in searching for new cytokines. To this end, a comprehensive database of gene expression in the human body, known as the Body Index of Gene Expression (BIGE), based on Affymetrix U133 2.0 microarrays (5, 6) was used. This database currently contains genome-wide expression data from 105 different human tissue/cell types, and includes lymphoid tissues and both resting and activated B and T lymphocytes. To identify novel genes of importance in the immune system, the BIGE database was searched for genes that were highly expressed in either lymphoid or myeloid cells or in tissues of the immune system (bone marrow, spleen, lymph nodes, thymus, tonsil) when compared to non-immune tissues. This screen yielded 511 genes associated with lymphoid tissues and 1569 associated with immune cells, and identified virtually all the immune system genes that have been identified and described in the last few decades, including most cytokine and chemokine genes. Importantly, also identified were 35 novel, poorly characterized genes, predicted to encode either transmembrane or secreted proteins that are highly expressed in the immune system. The inventors recently published an example of these genes, tetraspanin 33 (TSPAN33), a transmembrane protein that is expressed by activated B cells (7).

### B. IL40 is a Cytokine Produced by B Cells

The characterization of another one of these genes, C17orf99, which encodes a novel small secreted protein expressed in the fetal liver, bone marrow, and activated B cells, is described. The inventors have named this molecule interleukin-40 (IL40), since it has typical cytokine characteristics, including the fact that it is a small secreted protein produced by activated B cells. While IL40 is produced by B cells upon activation, its production is potentiated by Th2 cytokines (IL-4 and IL-13) or, TGFβ. Conversely, its production is inhibited by IFNγ, a Th1 cytokine. Il40^{-/-} mice display splenomegaly with increased numbers of B cells in the spleen. Additionally, Il40−/− mice have lower numbers of germinal center and total IgA secreting B cells in the intestinal peyer's patches, a site of IgA production. The connection between Il40 and IgA is further suggested by the observation that Il40 is induced upon the onset of lactation in the mammary gland. Finally, we show that Il40 transcription is elevated in splenocytes from MRLFas^{(lpr/lpr)} mice (a mouse model of SLE), suggesting that it may be involved in autoimmune disease

### Results

### A. Identification of a Novel Cytokine

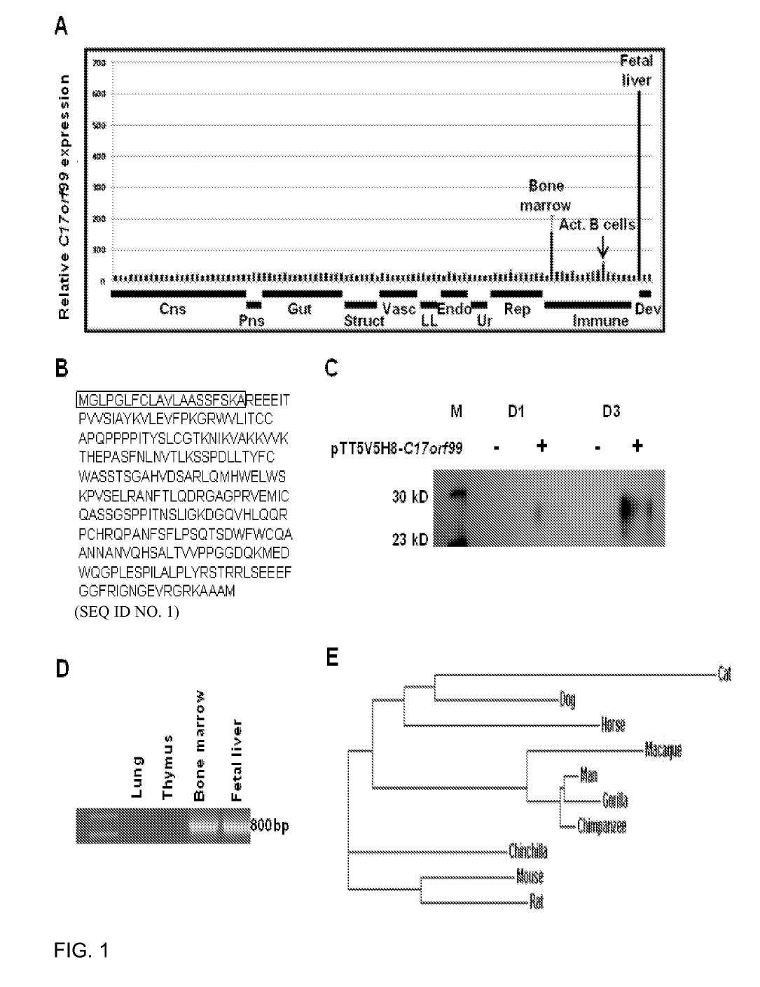

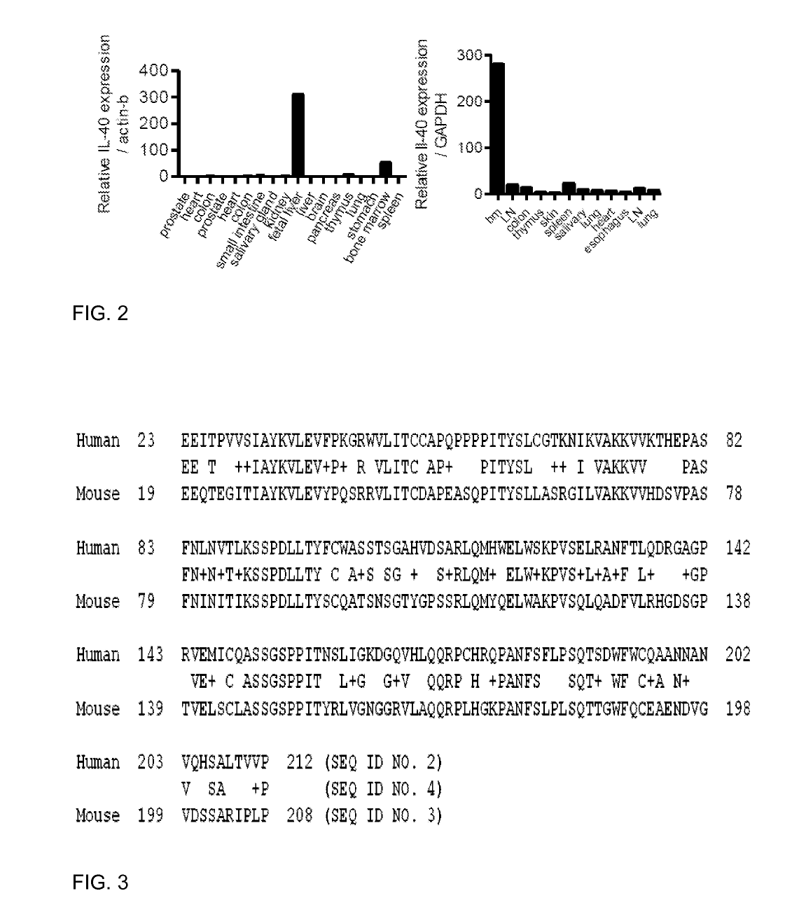

An unannotated immune system-associated gene (C17orf99) was first identified through the analysis of the BIGE database. C17orf99 mRNA is highly expressed in fetal liver, bone marrow and in B cells activated for 30 h with anti-CD40 +IL-4, with little or no expression elsewhere. A complete listing of all the tissues along with the mean intensities for C17orf99 expression is provided in Table 1. The human gene contains an open reading frame encoding a protein of 265 amino acids with a predicted N-terminal signal sequence of 20 amino acids (FIG. 1B) predicting a mature protein of 245 amino acids (˜27 KDa). A complete listing of the expression of IL40 in the BIGE database is provided in Table 1. Homologs were identified in mammalians, including primates, dogs and mice (6030468B19Rik, 72% protein sequence conservation), but absent in chicken and zebrafish (FIG. 2). The gene is named IL40 (FIG. 1), since it encodes a small secreted protein (8) produced in hematopoietic organs (homeostatic) (9) and by activated B lymphocytes (inflammatory) (2). The BIGE expression profile was confirmed using qRT-PCR analysis of human tissue RNAs (FIG. 2A) and, using an equivalent collection of mouse RNA samples, it was shown that the mouse homolog is also highly and specifically expressed in immune system-associated tissues and especially in bone marrow (FIG. 2B). A Pfam search indicates that IL40 does not belong to any current known cytokine families (data not shown), indicating that IL40 is a novel cytokine produced in hematopoietic organs and activated B cells.

TABLE 1
Complete listing of mean intensity values
of IL40 expression from BIGE database

| | |
|---|---|
| Vena_cava | 24.6 |
| Trachea | 17.6 |
| Bronchus | 19.3 |
| Lung | 18.3 |
| Adrenal_gland_cortex | 15.5 |
| Pancreas | 24.7 |
| Pituitary_gland | 19.8 |
| Thyroid_gland | 16.6 |
| Kidney | 19.0 |
| Kidney_cortex | 17.1 |
| Kidney_medulla | 19.5 |
| Urethra | 16.4 |
| Ovary | 16.1 |
| Testes | 16.5 |
| Fallopian Tube | 22.5 |
| Uterus | 20.7 |
| Myometrium | 18.9 |
| Endometrium | 26.2 |
| Cervix | 19.9 |
| Vagina | 21.5 |
| Vulva | 20.4 |
| Mammary gland | 18.7 |
| Nipple_cross_section | 22.7 |
| Penis | 20.3 |
| Prostate gland | 15.3 |
| Bone_marrow | 156.0 |
| Thymus_gland | 27.5 |
| Lymph_node | 26.0 |
| Spleen | 21.5 |
| Tonsil | 28.2 |
| PBMC_media_BB | 18.4 |
| PBMC_PMA + ionomycin | 20.0 |
| Monocytes_resting-30h | 22.4 |
| Monocytes_LPS + FNg-30h | 26.8 |
| B cells resting-30h | 31.9 |
| B_cells_antiCD40 + IL4-30h | 52.0 |
| T_cells_resting-30h | 23.6 |
| T cells antiCD3-30h | 19.1 |
| CD4+ | 20.3 |
| CD4 + antiCD3 + antiCD28 | 19.5 |
| CD8+ | 19.8 |
| CD8 + antiCD3 + antiCD28 | 17.6 |
| Fetal_liver | 608.7 |
| Fetal_brain | 18.7 |
| Placenta | 20.6 |

† Table obtained from BIGE database organized according to tissue and organ system. Mean intensity values of IL40 expression is listed (n = 8).

Prior to this study, C17orf99 was identified as a secreted protein in a survey of genes with predicted signal sequences (10). In order to verify that IL40 is secreted, human IL40 cDNA was cloned from human 2E2 B cells, a Burkitt's lymphoma model of B cell activation and differentiation (11), and inserted in-frame into the cloning site of the pTT5 (12) resulting in a recombinant gene encoding a fusion protein with a C-terminal 8× His tag. HEK 293 cells were then transfected with the pTT5-1140 construct, or empty vector as a control, and day 1 and day 3 supernatants collected and analyzed for the presence of recombinant IL40 protein. Western blot analysis of affinity purified supernatants using anti-His antibody detected an approximately 27 kD protein only in cells transfected with the pTT5-1140 construct, (FIG. 1C), confirming that IL40 is a secreted protein.

### B. IL40 is Produced by Activated B Cells

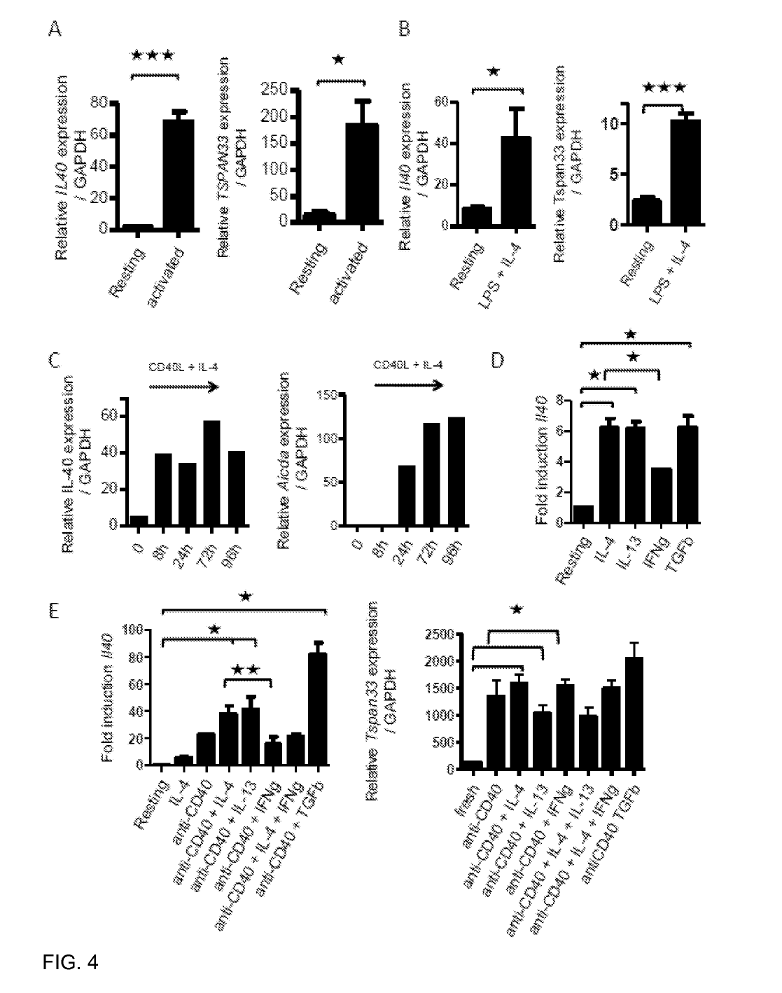

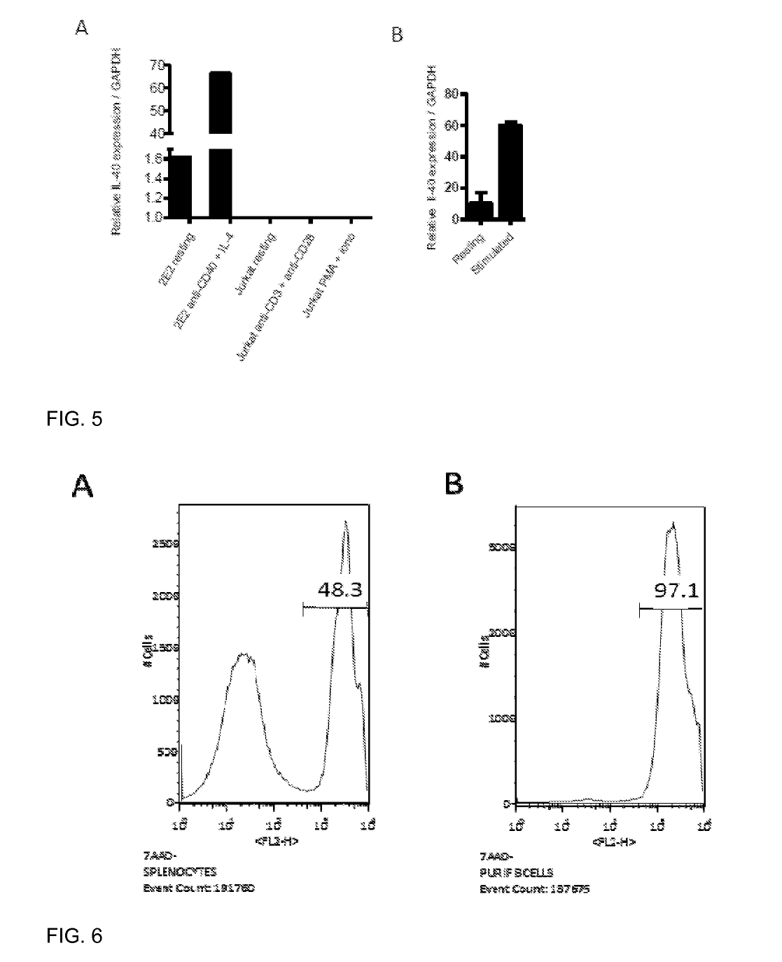

Since many cytokines, such as IL-2, IL-7, and IL-15, have functions in both lymphocyte ontogeny and activation (13), the inventors hypothesized that IL40 may also be involved in these processes. Its expression pattern in the BIGE database indicates that IL40 expression is increased when B cells are stimulated with CD40L+IL-4. To confirm this the transcription of IL40 in human B cells purified from PBMCs under resting or activating conditions was measured, with anti-CD40+IL-4 for 24 hours (FIG. 4A), and compared to TSPAN33 expression (a marker of B cell activation the inventors have recently described) (7). IL40 transcription was upregulated over 50 fold (p=0.002) along with TSPAN33 (p=0.01), indicating that IL40 is expressed by activated human B cells. These experiments were repeated using human 2E2 and Jurkat (T cell line), under resting or activating conditions (anti-CD40 mAb for 2E2 activation and anti-CD3+anti-CD28) (FIG. 5A). IL40 transcription was only induced in activated 2E2 cells. Similar results were obtained with mouse splenocytes activated with LPS+IL-4 (FIG. 4B) and A20-2J murine B cells stimulated under the same conditions (FIG. 5B). To measure the kinetics of IL40 transcription, Cd19⁺ B cells from C57BL/6 mice (FIG. 6) were stimulated with CD40L+IL-4 for 8, 24, 72, and 96 h and Il40 and Aicda (gene encoding AID, an enzyme involved in immunoglobulin class switching in activated B cells (14)); gene expression was measured by qRT-PCR (FIG. 4C). IL40 mRNA was upregulated within 8 hours and its expression remained elevated over 96 hours.

Since activated B cells have been shown to secrete Th1 type (from Be1) or Th2 (from Be2) cytokines depending on the stimuli (15) we sought to determine whether IL40 expression was modulated by cytokines as well.

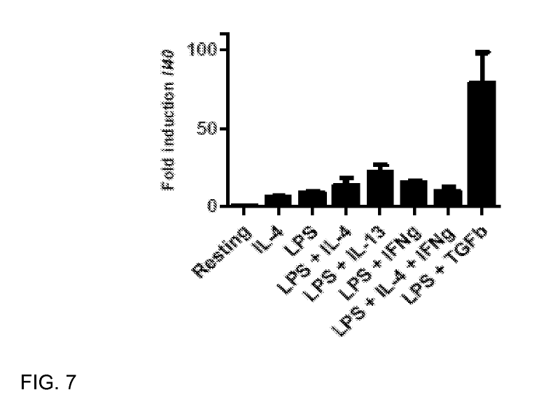

CD40 (FIG. 4E) or TLR4 (FIG. 7) stimuli were combined with IL-4, IL-13, IFNγ, or combinations in mouse splenocytes (FIG. 4D). IL40 transcription was induced upon Th2 cytokine stimuli (IL-4 and/or IL-13, ˜8 fold, while IFNγ (a Th1 cytokine) only increased it 3 fold. Anti-CD40 stimulation increased IL40 transcription over 20 fold. Interestingly, Th2 cytokines (IL4 or IL13), synergized the production of IL40 almost 2 fold higher than anti-CD40 stimulation alone, while IFNγ reduced the expression of IL40 to half the levels observed by anti-CD40 alone. Moreover, stimulation with anti-CD40+TGFβ induced the highest levels of IL40 production (FIG. 4E). The inventors conclude that IL40 production is induced upon anti-CD40 stimulation and synergized by Th2 cytokines and TGFβ, and is inhibited by IFNγ.

### C. IL40^{-/-} Mice Have Defects in B Cell Homeostasis

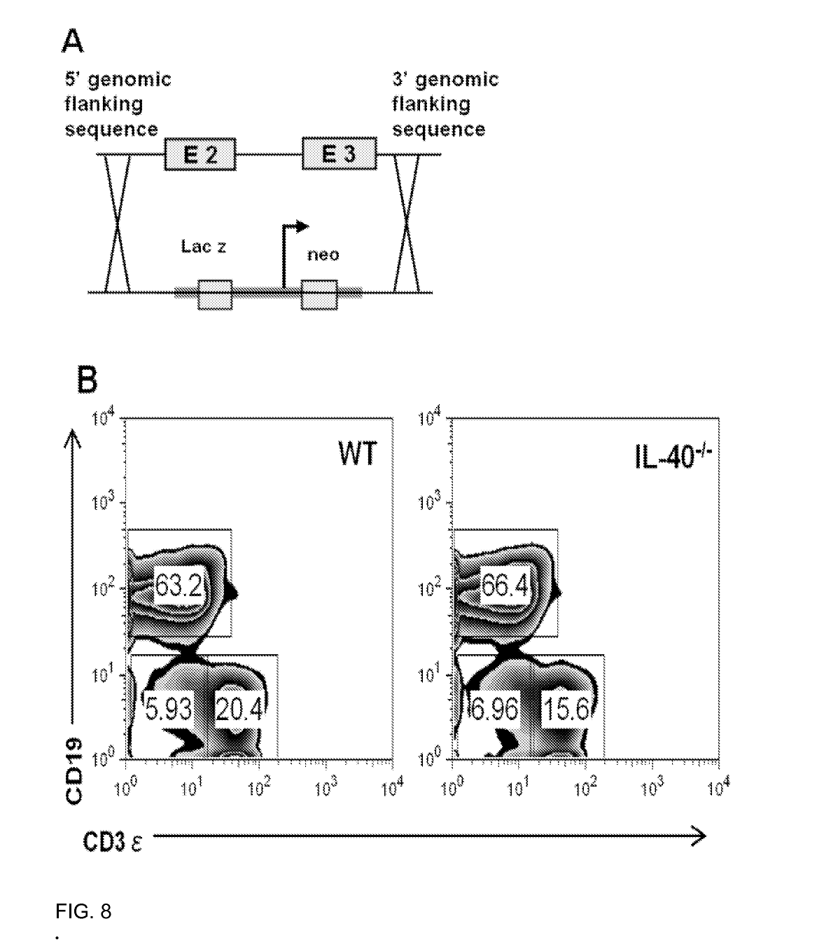

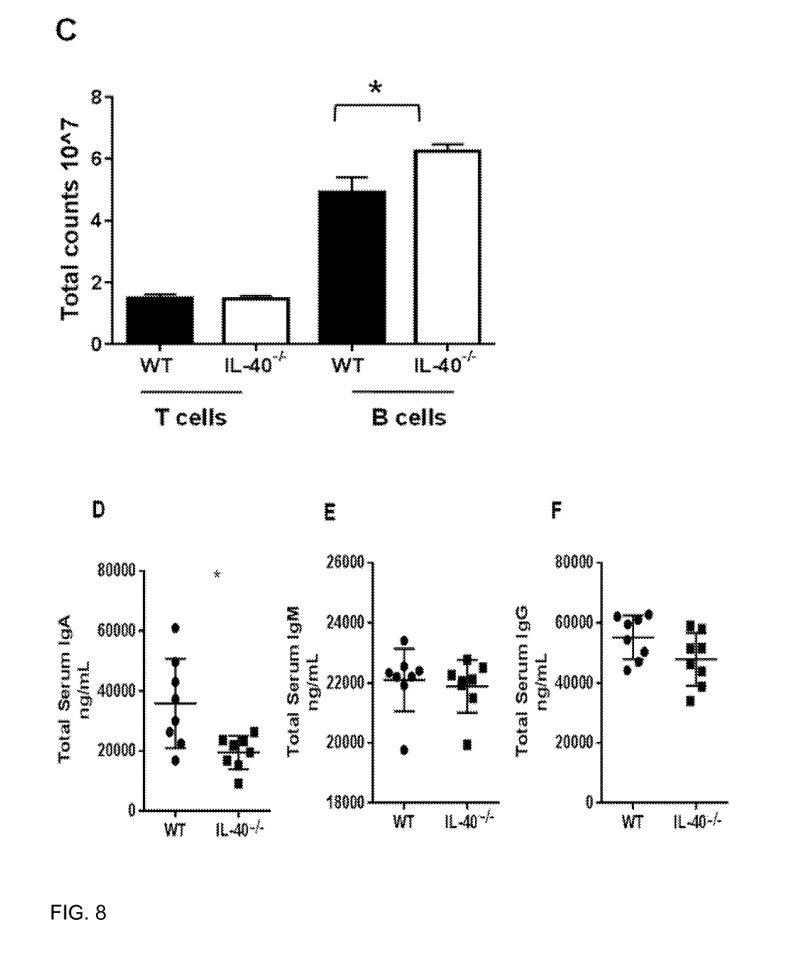

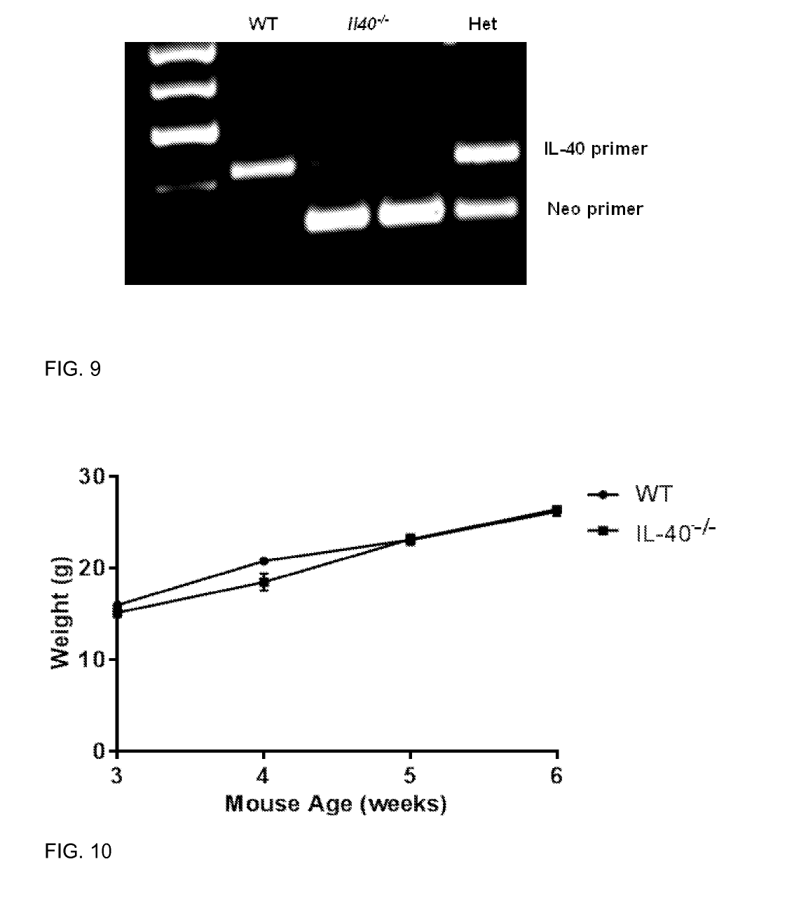

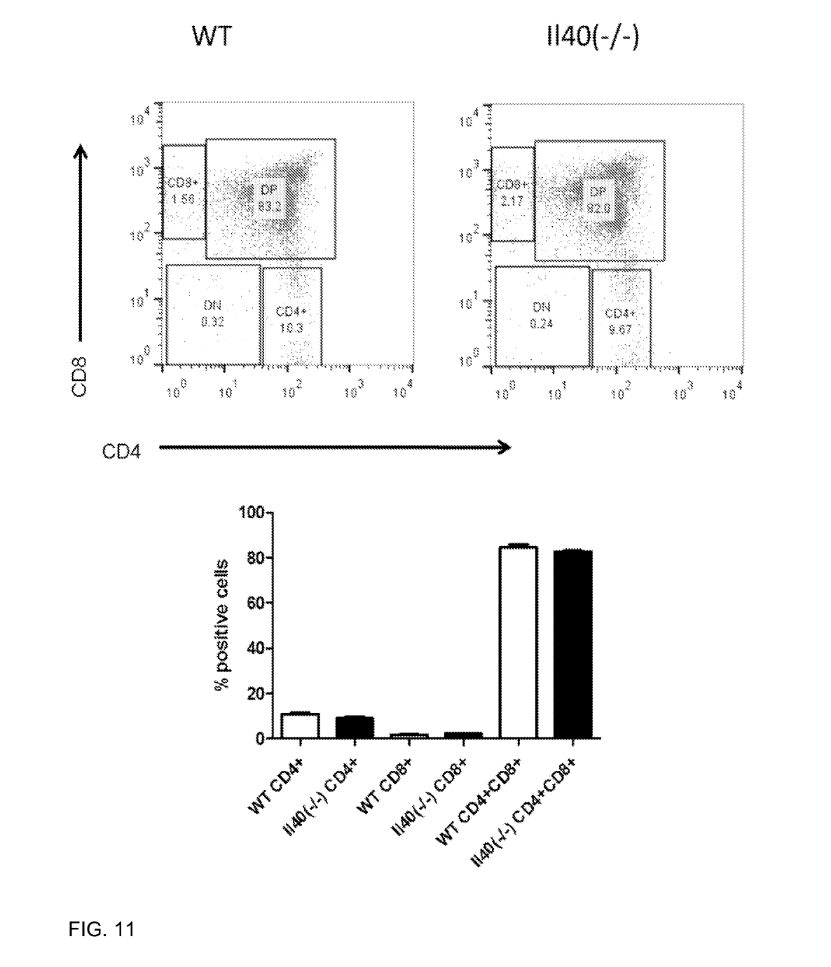

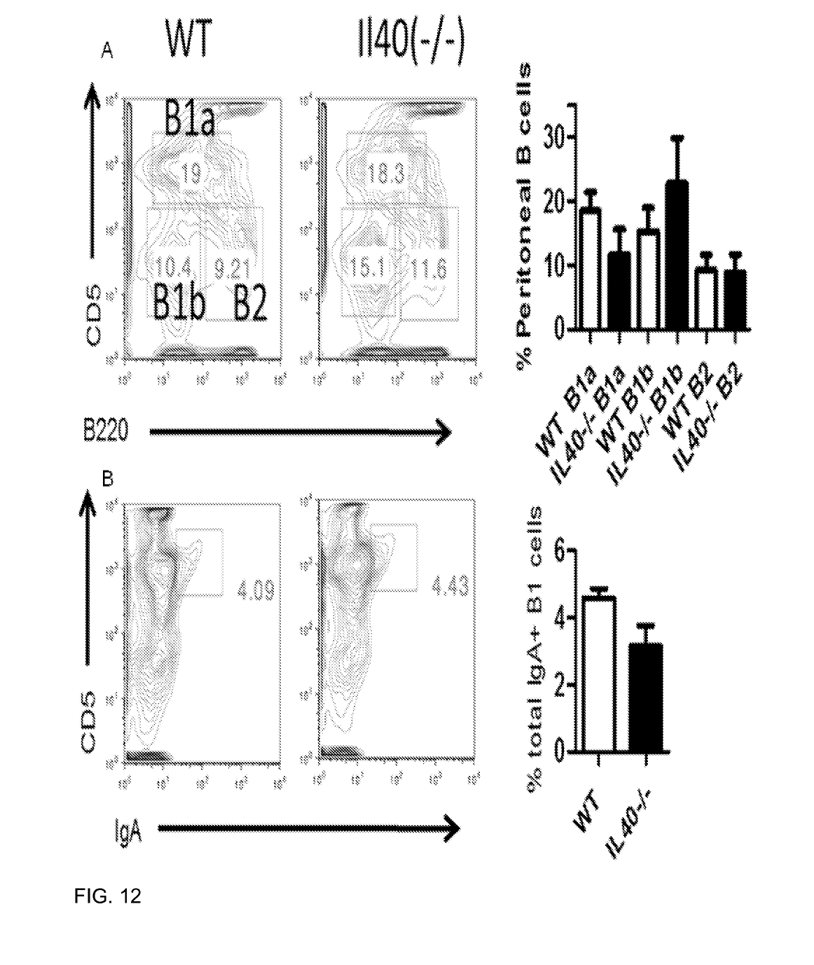

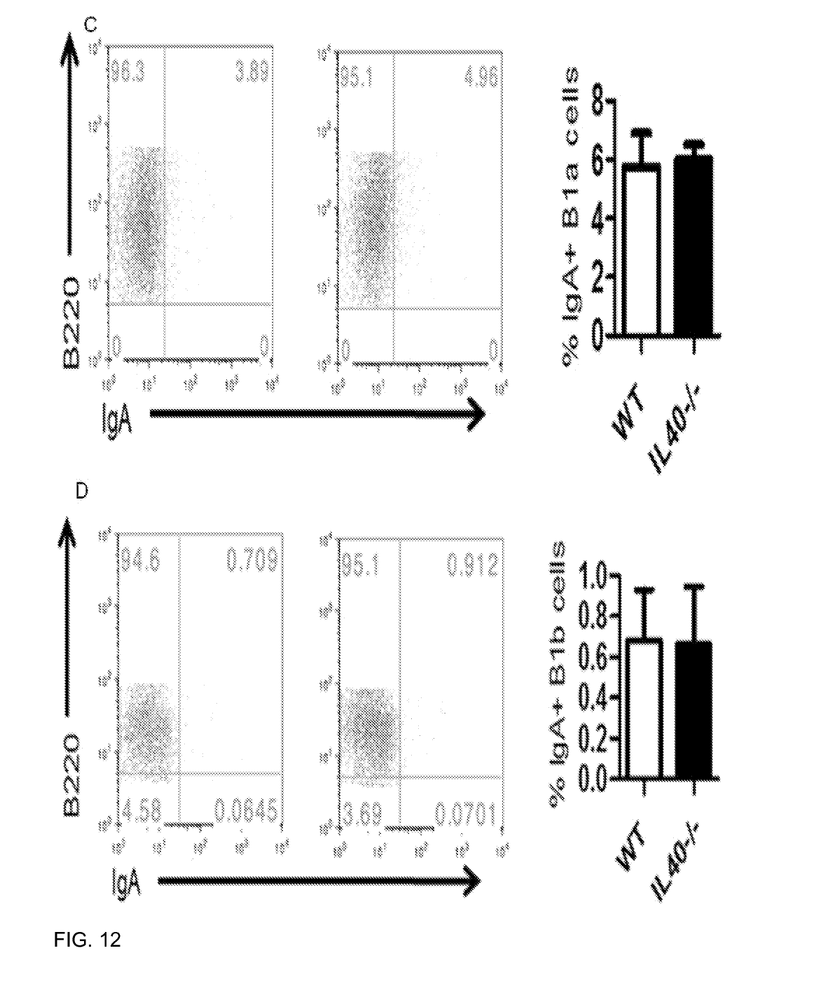

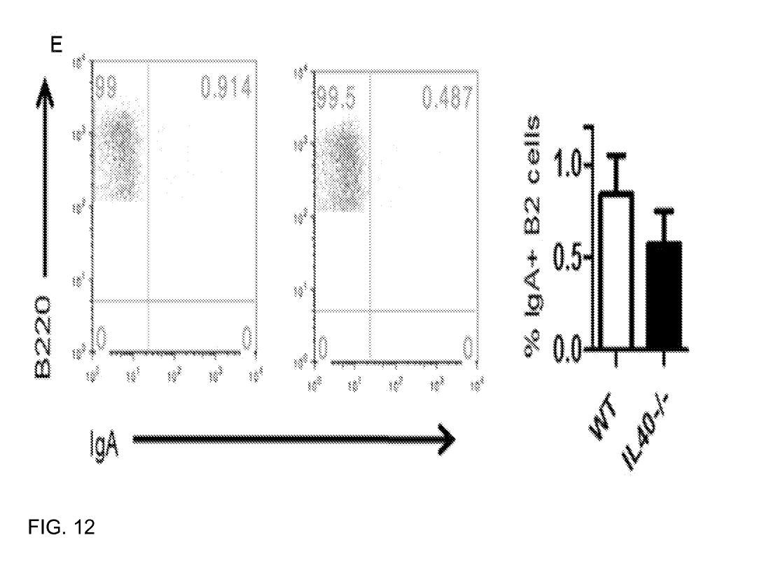

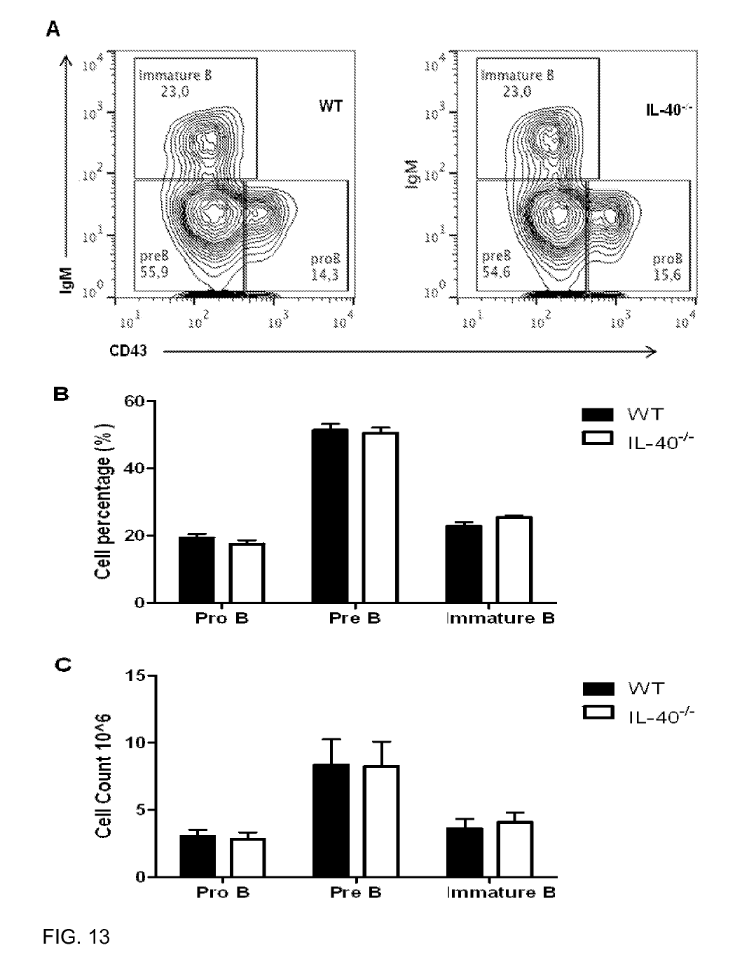

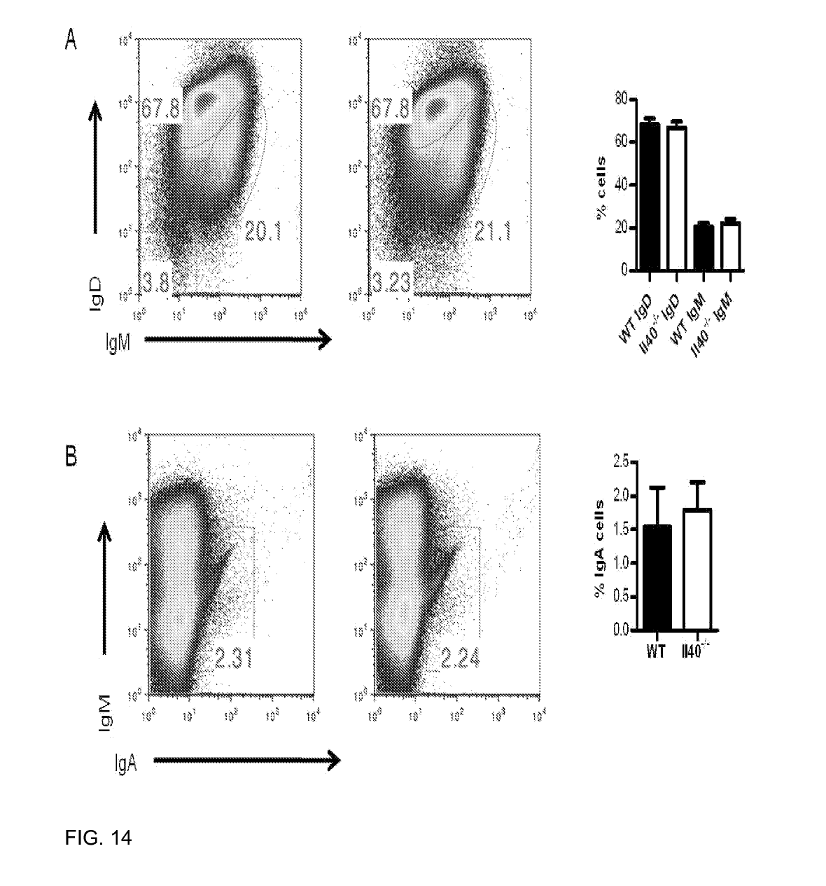

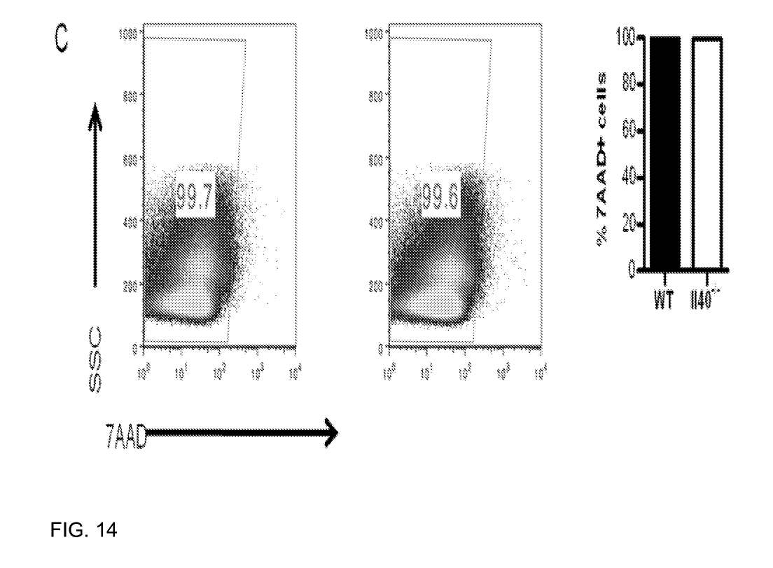

To further characterize the biological activity of IL40, we obtained a mutant mouse strain with a targeted deletion of the IL40 (6030468B19Rik) gene (FIG. 8a, see FIG. 9 for genotyping confirmation). IL40−/− mice are viable and fertile and have no defects in weight gain or body size (FIG. 10). T cell subsets (FIG. 11), B1/B2 cell ratios (FIG. 12), B cell maturation of plasma and germinal center cells (FIG. 13), and pro/pre/immature B cell populations (FIG. 14), in the thymus, peritoneal cavity, spleen, and bone marrow, respectively, were compared and no significant differences were found between Wt and IL40−/− mice. However, by 6 weeks of age, Il40−/− mice developed splenomegaly, as length (0.0940±0.004 vs. 0.1360±0.0151 cm, p=0.005), mass (0.1018±0.005 vs. 0.1366±0.0147 g, p=0.0285), and total cell numbers (76.03±6.87 vs. 96.34±2.15*10̂6 cells, p=0.0224) were elevated in IL40−/− mice. When the ratio of CD19+ B cells to T cells (CD3 +) was compared, IL40−/− mice were found to contain a higher proportion of B cells relative to T cells when compared to Wt mice although the difference was not statistically significant (4.4 vs. 3.4, p=0.086 (FIG. 8C). Additionally, IL40−/− mice exhibit elevated serum levels of IgG1 and IgA (FIG. 8F) over their wildtype (WT) counterparts (616 vs. 189 ug/mL, p=0.05 and 10 vs. 21 ug/mL, p=0.03, respectively). These data suggest possible alterations of B cell homeostasis in Il40−/− mice and suggest that Il40 may play a role in B cell differentiation/survival.

### D. IL40^{-/-} Mice Have Less IgA Producing Cells

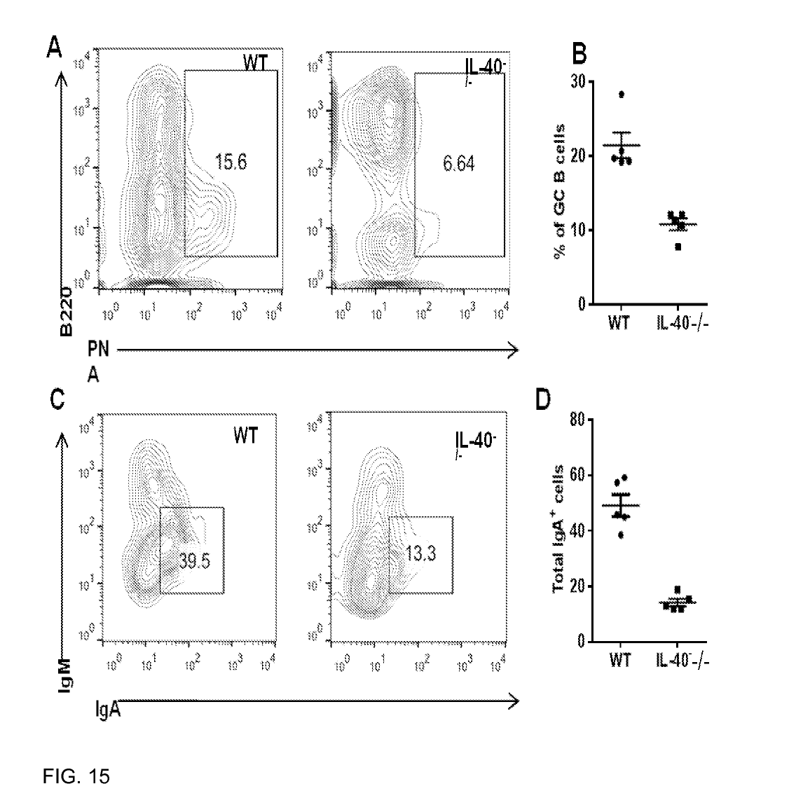

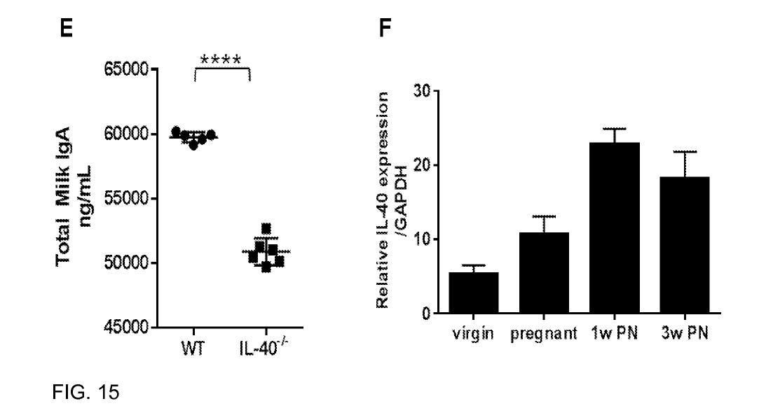

Since IL40^{-/-} mice appear to express elevated levels of serum IgA, an antibody isotype associated with mucosal immunity, the levels of plasma and germinal center cells in the Peyer's patches, gut associated lymphoid nodules that participate in immune surveillance of the digestive tract (15), were compared. Peyer's patches contain naturally occurring germinal center and IgA secreting plasma cells (16). Single cell suspensions were prepared from the Peyer's patches of WT and IL40^{-/-} mice and stained for the presence of germinal center cells (B220⁺PNA⁺) (FIG. 15a), IgA switched germinal center cells (PNA⁻B220^{hi}IgM^{lo}IgA⁺) (FIG. 15B), and total IgA switched cells (B220^{lo-hi}PNA⁺IgA⁻) (FIG. 15C), measured by flow cytometry (17-19). Il40^{-/-} mice had a greater than 2-fold reduction in germinal center cells (n=5, p=0.0005), 2.5-fold reduction in IgA switched germinal center cells (n=5, p=0.0001) and a 10 fold reduction in total IgA positive cells (PNA⁻ germinal center B220^{hi} and B220^{neg-lo} plasma cells) (n=5, p=0.0001). Additionally, IgA levels in the fecal pellets of Wt and Il40^{-/-} mice were measured by ELISA (FIG. 15D), to determine if the defect in IgA positive cells affects the level of IgA secreting in the mucosa. There was a 2-fold reduction in IgA levels in the fecal pellets of Il40^{-/-} compared to Wt mice (n=10, p=0.006). The IgA defect present in the IL40^{-/-} mice led us to explore whether Il40 is expressed in the mammary gland. As shown in FIG. 15E, IL40 expression is induced upon lactation in the mammary gland. Taken together, these observations suggest a role for IL40 in IgA responses.

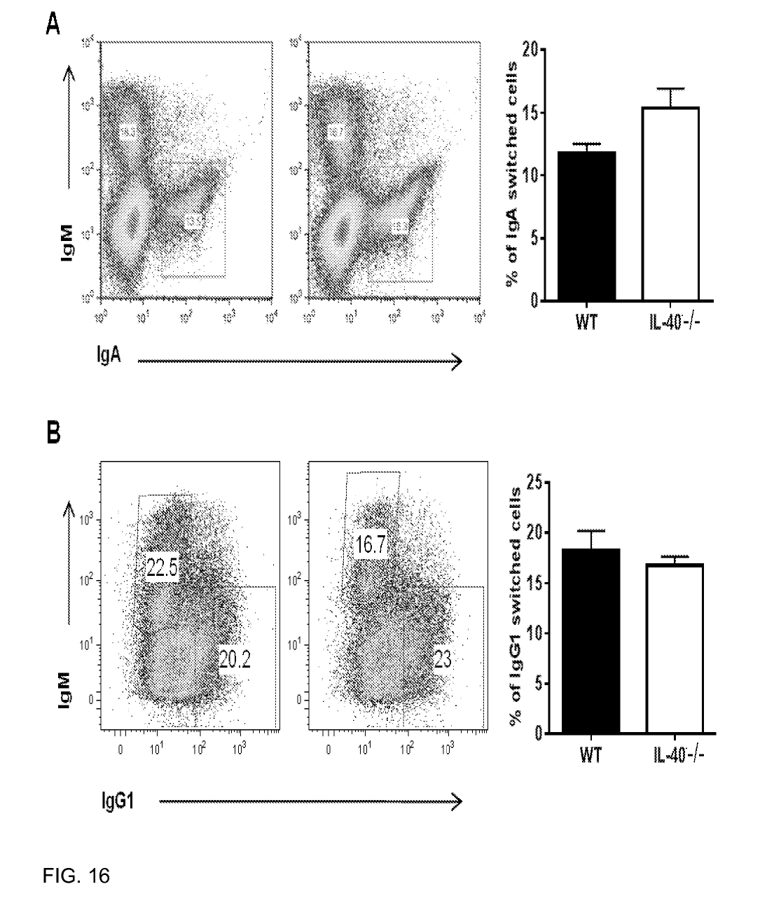

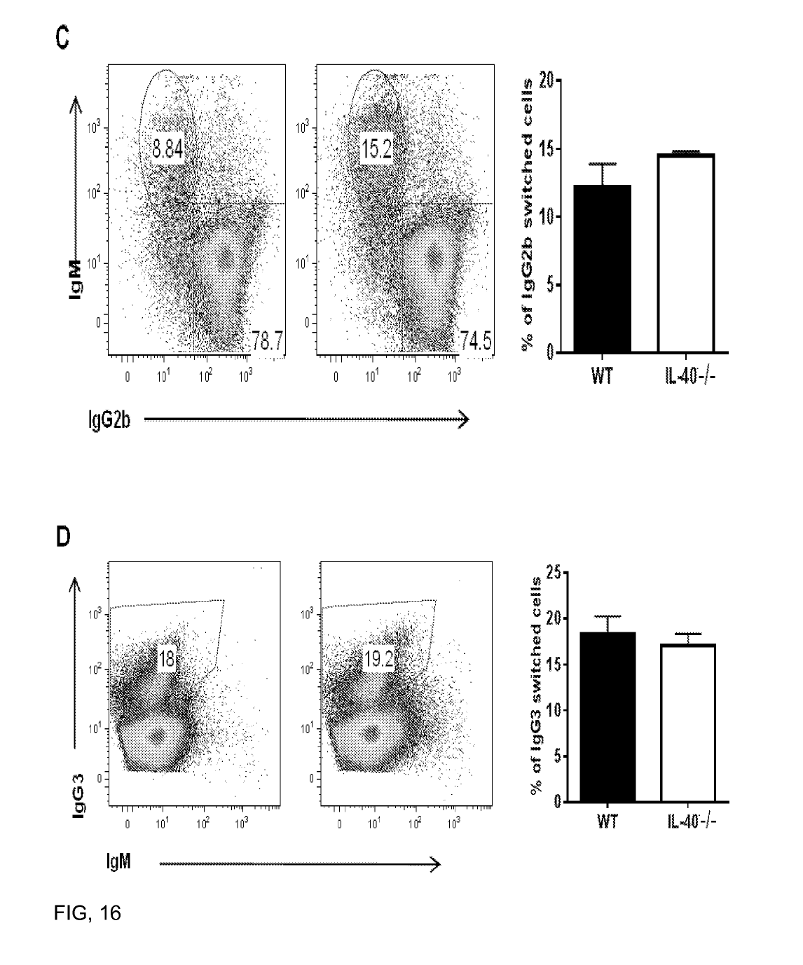

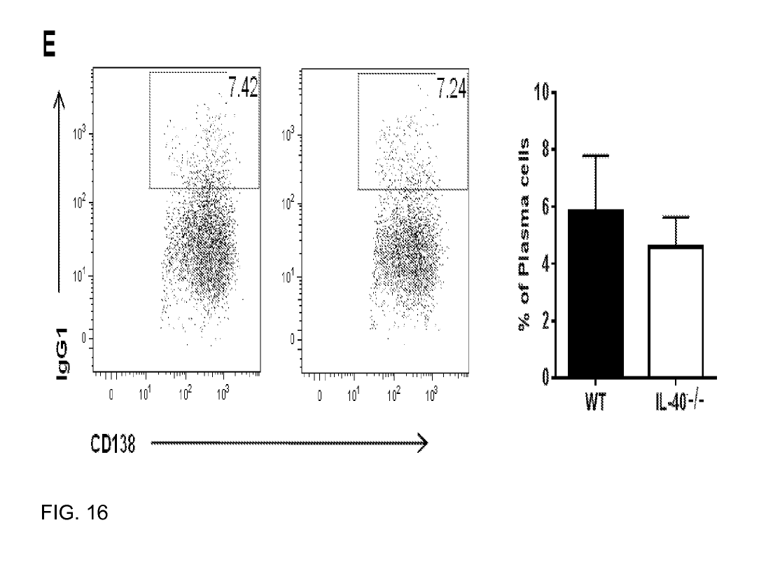

These changes can be attributed to either defects in class-switch recombination (CSR) mechanisms (20, 21) or to a defect in the maturation of the germinal center response (proliferation (22), survival (23), activation (24). In order to address the first possible mechanism, in vitro induction of CSR in WT and Il40^{-/-} B cells was performed and the resulting IgG1, IgG2a, IgG2b, and IgA, class switching was monitored using flow cytometry see (FIG. 16). There were no differences between Wt and Il40^{-/-} IgG1, IgG2a and IgG3 switched cells and only a slight increase in IgA switching in Il40^{-/-} B cells (p=0.03). The inventors therefore conclude that the paucity of IgA producing cells in the Peyer's patches of Il40^{-/-} mice is not due to an impairment of the ability to undergo CSR.

### Identification of IL40 Response Genes

The inventors then sought to explore in more detail the effects of IL40 on lymphocytes. To this end, global gene expression analysis on resting and LPS+IL-4 stimulated lymphocytes obtained from the lymph nodes of WT or Il40^{-/-} mice using genearrays was performed.

Two groups of genes profiles were characterized, genes that are upregulated in the WT vs. IL40^{-/-} mice (Table 2), under resting or activated conditions, and genes that are upregulated in the IL40^{-/-} mice vs. WT (Table 3). As expected, the top differentially expressed gene was IL40. Interestingly the 4^{th} top differentially expressed gene was synaptogyrin 2 (Syngr2). Synaptogyrin 2 belongs to the synaptogyrin family found in neurons of the central nervous system, but unlike its family members, Syngr2 is not expressed in the central nervous system. To determine the expression profile of SYNGR2, the BIGE database was analyzed for sites of SYNGR2 expression (FIG. 17, see Table 4 for top sites of SYNGR2 expression in BIGE). SYNGR2 was determined to be expressed by activated B cells, monocytes, and human colon.

TABLE 2
List of top 25 genes upregulated in WT vs IL40^{-/-} mouse splenocytes

| | | Wt vs KO R | WT vs KO A | |
|---|---|---|---|---|
| Gene Description | Gene Symbol | ratio | ratio | Notes |
| RKEN cDNA 6030468B19 gene | 6030468B19Rik | 5.23 | 7.84 | |
| predicted gene, 6664 | Gm6664 | 0.30 | 6.49 | Pseudogene |
| proline-rich protein BstNI subfamily 1 | Prb1 | 1.11 | 4.65 | |
| synaptogyrin 2 | Syngr2 | 3.65 | 4.51 | |
| predicted gene, 9312 | Gm9312 | 0.15 | 3.27 | Pseudogene |
| predicted gene, 24724 | Gm24724 | 1.11 | 3.05 | snRNA |
| histone cluster 1, H1a | Hist1h1a | 3.34 | 2.98 | |
| predicted gene, 23825 | Gm23825 | 0.74 | 2.96 | snoRNA |
| predicted gene 15807 | Gm15807 | 1.57 | 2.95 | pseudogene |
| predicted gene, 25153 | Gm25153 | 2.09 | 2.85 | snRNA |
| RKEN cDNA 4930457A20 gene | 4930457A20Rik | 1.17 | 2.75 | Known antisense |
| olfactory receptor 820 | Olfr820 | 0.77 | 2.70 | |
| microRNA 20b | Mir20b | 1.11 | 2.68 | |
| fibroblast growth factor 23 | Fgf23 | 1.09 | 2.62 | |
| microRNA 669o | mir669o | 1.20 | 2.60 | miRNA |
| killer cell lectin-like receptor famly lmember 1 | Klri1 | 1.73 | 2.58 | |
| predicted gene, 23516 | Gm23516 | 1.35 | 2.57 | snRNA |
| T cell receptor alpha variable 15N-1 | Trav15n-1 | 0.88 | 2.52 | |

† Table obtained from Affymetrix MOGENE 2.0 ST arrays, on resting and LPS + IL-4 induced lymphocytes from the lymph nodes of WT (n = 1) and IL-40 deficient mice.

TABLE 3
List of top 25 genes upregulated in IL40^{-/-} vs WT mouse splenocytes

| | | Wt vs KO R | Wt vs KO A | |
|---|---|---|---|---|
| Gene Description | Gene Symbol | ratio | ratio | Notes |
| vomeronasal 1 receptor 208 | Vmn1r208 | 6.61 | 1.38 | |
| RKEN cDNA 6030468B19 gene | 6030468B19Rik | 5.23 | 7.84 | KO target gene |
| predicted gene 4567 | Gm4567 | 4.28 | 0.56 | serine/threonine kinase-like |
| synaptogyrin 2 | Syngr2 | 3.65 | 4.51 | |
| killer cell lectin-like receptor subfamily G, member 1 | Klrg1 | 3.39 | 2.35 | |
| histone cluster 1, H1a | Hist1h1a | 3.34 | 2.98 | |
| SH2 domain protein 181 | Sh2d1b1 | 3.24 | 0.95 | |
| predicted gene, 22657 | Gm22657 | 3.15 | 0.76 | SnoRNA |
| olfactory receptor 917 | Olfr917 | 3.02 | 0.70 | |
| small nucleolar RNA, H/ACA box 34 | Snora34 | 2.96 | 1.13 | |
| ribosomal L24 domain containing 1 | Rsl24d1 | 2.88 | 1.24 | |
| predicted gene, 25291 | Gm25291 | 2.87 | 1.29 | SnoRNA |
| immunoglobulin heavy variable V3-3 | Ighv3.3 | 2.86 | 1.30 | |
| S100 calcium binding protein A6 (calcyclin) | S100a6 | 2.86 | 1.56 | |
| Predicted gene, 26419 | Gm26419 | 2.77 | 1.03 | SnoRNA |
| hemoglobin, beta adult major chain | Hbb-b1 | 2.72 | 2.10 | |
| small nucleolar RNA, H/ACA box 15 | Snora15 | 2.71 | 1.94 | SnoRNA |
| Predicted gene, 24727 | Gm24727 | 2.68 | 0.91 | SnoRNA |
| Clone MBI-31 H/ACA box snoRNA, partial sequence | AF357391 | 2.62 | 1.02 | SnoRNA |
| Predicted gene, 25803 | Gm25803 | 2.62 | 1.17 | SnoRNA |
| pentatricopeptide repeat domain 3 | Ptcd3 | 2.61 | 1.18 | |
| Predicted gene | Gm | 2.56 | 1.19 | SnoRNA |
| Predicted gene | Gm | 2.54 | 1.06 | SnoRNA |
| Predicted gene | Gm | 2.53 | 1.02 | SnoRNA |

† Table obtained from Affymetrix MOGENE 2.0 ST arrays, on resting and LPS + IL-4 induced lymphocytes from the lymph nodes of WT (n = 1) and IL-40 deficient mice.

TABLE 4
Top 10 sites of synaptogyrin 2 expression in humans

| | Mean |
|---|---|
| Tissue | intensity |
| B_cells_antiCD40 + IL4-30h | 2998 |
| B_cells_resting-30h | 1299 |
| Monocytes_resting-30h | 1224 |
| Colon | 803 |
| Prostate_gland | 767 |
| Tonsil | 706 |
| Small_intestine | 694 |
| PBMC_media_BB | 685 |
| Salivary_gland | 682 |
| Monocytes_LPS + IFNg-30h | 671 |
| Tongue | 661 |
| Trachea | 658 |
| CD4+_antiCD3 + antiCD28 | 647 |
| T_cells_antiCD3-30h | 647 |
| Lung | 641 |
| Tongue_superior | 630 |

† Table obtained from BIGE database of Synaptogyrin 2 expression

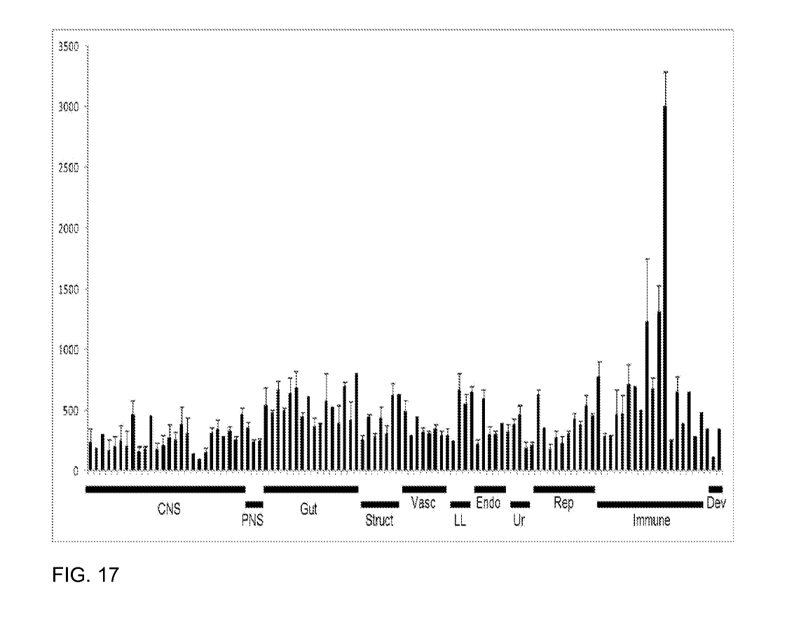

Lastly, while it has been determined that IL40 is produced by activated B cells, the target cells of IL40 are not known. To determine whether IL40 acts on B cell or T cell gene expression, 50 genes considered “genes involved in B cell activation/differentation,” such as CD81, CD86, Ms4a1 (CD20), IL-4, and Stat6, and 38 genes considered “genes involved in T cell activation/differentiation,” such as CD3 (3 genes), Cxcr4, Il2α, and IFNγ, were compared between the WT and Il40^{-/-} mouse samples (Table 5). If IL40 acts on T cells, then the absence of IL40 (in the Il40^{-/-} samples) should affect the T cells but not the B cells; conversely, if IL40 acts on B cells, then we would expect a difference in B cell activation genes (FIG. 18). Additionally, if IL40 acts on both cell types, then both gene sets should change. In the resting state, only B cell expressed genes were differentially expressed between WT and Il40^{-/-} mice, although this difference was not statistically significant (p=0.09), (probably due to the smaller number of B cells in the lymph node samples used compared to T cells). However, in the LPS+IL-4 stimulated samples, B cell, but not T cell genes, were the only ones differentially expressed (p=0.018). These results strongly suggest that the IL40 responding cells are B cells.

TABLE 5
Gene expression in wild type and IL40−/− samples.

| | Wt vs KO R | Wt vs KO A |
|---|---|---|
| Gene Symbol | ratio | ratio |
| Cd28 | 0.87 | 0.96 |
| Cd4 | 0.72 | 0.66 |
| Cd40 | 1.21 | 1.18 |
| Cd40lg | 1.13 | 1.04 |
| Il10 | 1.26 | 0.89 |
| Cr2 | 1.29 | 1.08 |
| Icosl | 1.32 | 1.23 |
| Igbp1 | 1.04 | 0.89 |
| Ms4a1 | 1.31 | 1.32 |
| Rgs1 | 1.04 | 0.68 |
| Sla2 | 0.77 | 0.71 |
| Cd81 | 0.91 | 0.93 |
| Cdkn1a | 0.89 | 1.05 |
| Prkcd | 1.09 | 1.15 |
| Ptprc | 0.94 | 1.12 |
| Bad | 1.06 | 0.86 |
| Clcf1 | 1.17 | 1.34 |
| Hdac5 | 1.05 | 1.02 |
| Hdac7 | 0.6 | 0.67 |
| Pik3r1 | 0.87 | 0.89 |
| Cd86 | 1.18 | 1.07 |
| Egr1 | 0.96 | 0.79 |
| Stat6 | 1.05 | 1.13 |
| Ptpn2 | 1.08 | 1 |
| Bcl2l1 | 0.36 | 0.63 |
| Ighg | 1.05 | 1.19 |
| Ighg | 1.24 | 1.12 |
| Ighg | 0.9 | 1 |
| Ighg | 1.09 | 1.57 |
| Ighg | 1.08 | 1.16 |
| Ighg | 1.21 | 0.97 |
| Ighg | 1.36 | 1.28 |
| Ccl22 | 0.5 | 0.44 |
| Nfatc1 | 1.07 | 1.05 |
| Nfatc2 | 1.07 | 1.2 |
| Ikzf3 | 0.96 | 1.04 |
| Spib | 1.22 | 1.28 |
| Tcf3 | 1 | 1.03 |
| Pou2af1 | 1.39 | 1.28 |
| Il21r | 1.03 | 1.13 |
| Cd44 | 1.31 | 1.19 |
| Fas | 0.59 | 0.61 |
| Fasl | 1.33 | 1.04 |
| Cblb | 1.1 | 1.12 |
| Cd1d1 | 0.89 | 0.85 |
| Cd3g | 0.88 | 0.84 |
| Cd8a | 0.58 | 0.52 |
| Dock2 | 0.99 | 1.07 |
| Irf4 | 1.42 | 1.4 |
| Prkcq | 1.03 | 0.98 |
| Sit1 | 0.67 | 0.56 |
| Vav1 | 1 | 0.97 |
| Was | 0.96 | 0.94 |
| Ccnd3 | 0.73 | 0.7 |
| Cd3e | 0.83 | 0.81 |
| Cxcl12 | 0.84 | 1.29 |
| Cxcr4 | 1.08 | 0.95 |
| Glmn | 1.13 | 1 |
| Il12b | 0.94 | 0.52 |
| Il18 | 1.04 | 1.2 |
| Il2ra | 1.21 | 1.16 |
| Cd74 | 1.09 | 1.13 |
| H2-Aa | 1.37 | 1.29 |
| Hsp90aa1 | 1.05 | 1.13 |
| Il27 | 0.8 | 0.94 |
| Jag2 | 0.82 | 1.02 |
| Socs5 | 1.3 | 1.01 |
| Wwp1 | 1 | 0.95 |
| Cd93 | 1.02 | 1.12 |
| Tlr6 | 1.01 | 1.08 |
| Impdh1 | 0.65 | 0.74 |
| Flt3 | 0.91 | 0.88 |
| Nfatc1 | 1.07 | 1.05 |
| Nfatc2 | 1.07 | 1.2 |
| Ikzf3 | 0.96 | 1.04 |
| Spib | 1.22 | 1.28 |
| Tcf3 | 1 | 1.03 |
| Pou2af1 | 1.39 | 1.28 |
| Il21r | 1.03 | 1.13 |
| Cd44 | 1.31 | 1.19 |
| Fas | 0.59 | 0.61 |
| Fasl | 1.33 | 1.04 |
| Cblb | 1.1 | 1.12 |
| Cd1d1 | 0.89 | 0.85 |

### IL40 is Upregulated in Systemic Lupus Erythematosus

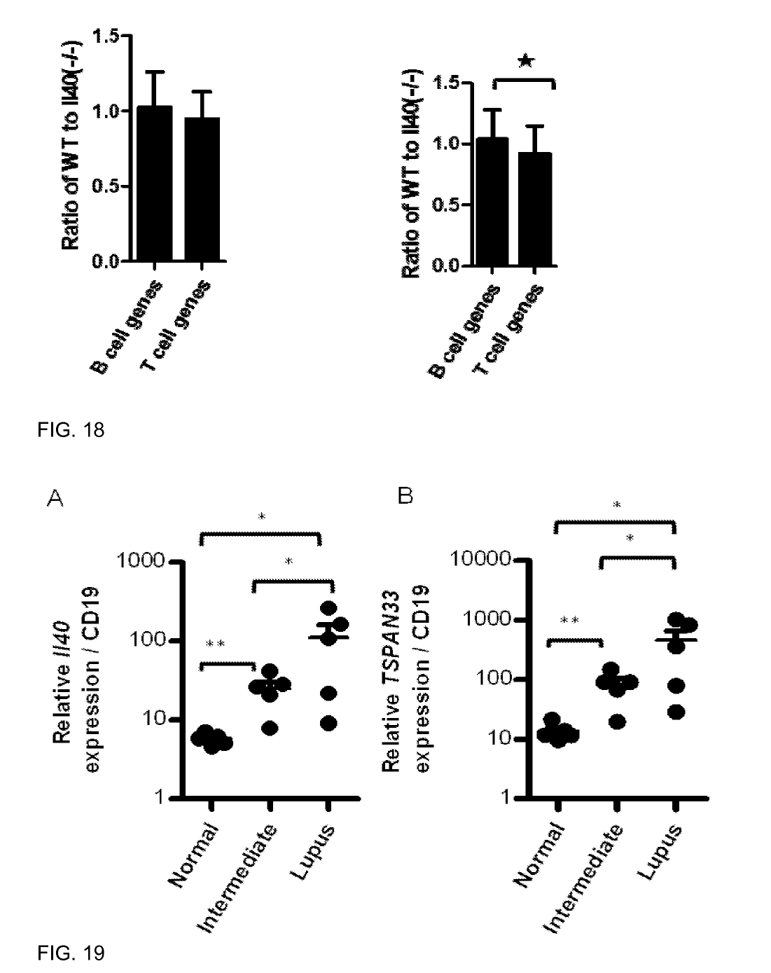

Since levels of known B cell cytokines, such as IL-6, IL-21 and BLyS are often dysregulated in autoimmune diseases (7), the mRNA expression of Il40 in splenocytes from the MRL/Fas^{lpr/lpr} mouse model of systemic lupus erythematosus (SLE) was measured. In this model, mice show increasing levels of systemic autoimmunity with production of anti-dsDNA (double stranded DNA) antibodies and immune glomerulonephritis, both of which are hallmarks of SLE. Young mice appear normal and at 9 weeks exhibit no visible pathology. By week 24, intermediate lupus symptoms with lymphoadenopathy and some skin lesions develop, and by week 36, full lupus symptoms are apparent often leading to death (25). IL40 mRNA expression (FIG. 19) was found to increase. The increase in IL40 levels detected in a mouse model suggests that dysregulation of IL40 may be involved in SLE pathology and may represent a target for treatment of the disease.

While there has been significant progress in the functional characterization of human genes, for a large number of them there is little or no information. Indeed, while the consensus coding sequence (CCDS) (26, 27) project lists 18,673 GeneIDs (Release 14), only 13669 of these have descriptive names (HUGO Gene Nomenclature Committee) (28). Thus, over 5,000 CCDS entries do not have useful names and most remain poorly characterized. Another complication preventing their study is the lack of reagents to study them. Here, the inventors report on the identification of a novel cytokine, which is called IL40, encoded by the uncharacterized gene C17orf99. C17orf99 was initially identified as part of a set of 86 genes identified through analysis of the BIGE database of human gene expression (29) whose expression pattern is associated with either organs of cells of the immune system with little or no expression elsewhere in the body, and that are predicted to encode either transmembrane or secreted proteins. The inventors have recently reported the identification of a novel B cell activation molecule (TSPAN33) which was also identified using this approach (7). IL40/C17orf99 is the second gene the inventors are reporting as part of this analysis.

The identification of protein encoded by C17orf99 as the novel cytokine interleukin-40 (IL40) is based on several factors. Firstly, it is a small secreted protein whose expression is restricted to several tissues and cells of the immune system—similar to the expression profiles for many other known cytokines The inventors began exploring the expression of IL40 in the bone marrow and found that, in contrast to other cytokines, it was expressed by the lymphoid and not the bone marrow stromal cells. The small but significant expression of IL40 by activated peripheral blood B cells in the BIGE database further suggested that it was a B cell product. IL40 encodes a small secreted protein, and its expression is induced in B cells cultured under proinflammatory conditions, and is modulated by various cytokines Taken together, the inventors conclude that C17orf-99 encodes a novel B cell derived cytokine

Until recently, most known cytokines, even those recently characterized, belong to known cytokine families. IL37, for example, is a member of the interleukin1 family. This facilitated their identification. In contrast, IL40 does not belong to any known cytokine family. The inventors have identified homologs in mammals, but not in chicken or zebrafish. This indicates that its function may be restricted to the mammalian immune system.

Indeed, the inventors have determined that IL40 is induced in B cells, upon activation with anti-CD40 mAb (or CD40L, or LPS), in the presence of Th2 cytokines (IL-4 and IL-13), but not IFNγ, a Th1 cytokine IL40 is most significantly induced by activation in the presence of TGFβ, however. It is possible that some of the functions previously attributed to TGFβ on B cell function may be indirectly mediated by IL40. TGFβ simultaneously inhibits B cell proliferation (30) and CSR towards IgG1, IgG2a, IgG3, and IgE, while inducing CSR towards IgA and IgG2b.

As B cell conditional knockouts of TGFβ (31) or TGFR (30) (or components of the TGFR, i.e. Smad2^{-/-} (32)) mice have defects in IgA production in the peyer's patches, but an increase in the frequency and numbers germinal center cells and CSR towards all other isotypes. Although Il40^{-/-} mice have a defect in IgA producing cells in the Peyer's patches, they also have decreased germinal center cells, suggesting that the mechanisms of this defect are separate from TGFβ or TGFR deficient mice. Additionally, endogenous TGFβ is necessary for CSR towards all isotypes during in vitro splenocyte induction of CSR, as inhibiting endogenous TGFβ reduces CSR of all isotypes (33), yet B cells from Il40^{-/-} mice did not have a defect in CSR during the in vitro induction with LPS and Th1 or Th2 cytokines, although there was a slight increase in IgA switching. It is important to note that a defect in GC and IgA producing cells could also be caused by defects in the GC response, such as activation (CD40 or B7 deficient mice) (34), proliferation (Ccnd3^{-/-} mice) (22), survival (Pdcd1Ig2^{-/-}, CD274^{-/-}Pdcd1Ig2^{-/-} and Pdcd1^{-/-} mice) (35), or migration into the lamina propria (S1P inhibition by FTY720) (36). However, none of these defects resemble the phenotype of the Il40^{-/-} mice that have increased B cell numbers in the spleen, but lower number of germinal center and IgA secreting cells in the Peyer's patches. We also detected lower IgA in the feces of the Il40^{-/-} mouse. TGFβ strongly potentiates the production of Il40 by activated B cells, and Il40 is induced upon the onset of lactation in the mammary gland. Taken together, these observations indicate that Il40 is involved in the differentiation of B cells towards IgA responses.

Recently, cytokines produced by B cell subsets have been recognized as key regulators of immune modulation, as B cells were shown to have reciprocal interactions with T cells in skewing pro or anti-inflammatory responses through secreted cytokines (37). The discovery of a novel cytokine produced by activated B cells is highly significant, as tailoring specific cytokine interactions has gained popularity as a therapeutic strategy in the treatment of autoimmune diseases, such as rheumatoid arthritis (RA) and SLE (38, 39). Furthermore, B cells have been recognized to be involved in many human autoimmune diseases, therefore identifying a novel cytokine produced by B cells will contribute to our understanding of B cell mediated pathology.

Here the inventors describe the first report of IL40, a novel cytokine produced exclusively by activated B cells in the periphery. Since there are no previous reports on IL40, its exact pathways and signaling mechanisms are not known. The inventors believe that IL40 may function in autocrine signaling by activated B cells, since microarray analyses revealed that mostly B cell genes were altered when comparing WT and Il40^{-/-} mouse lymph nodes. The discovery of a novel cytokine involved in B cell differentiation is also important in understanding the involvement of B cell cytokines in modulating the immune response. Additionally, IL40^{-/-} transcription is elevated in mice and humans with SLE, suggesting that the dysregulation of IL40 may be involved in autoimmune disease.

### Methods:

### B Cells, CSR and Plasma Cell Differentiation

Spleen cells were resuspended in RPMI with FBS (10%), 50 mM β-mercaptoethanol and 1× antibiotic-antimycotic mixture (15240-062; Invitrogen Corp.) at 37° C. in 48-well plates and stimulated with the following reagents: LPS (5 μg/ml) from Escherichia coli (055:B5; Sigma-Aldrich) plus TGF-β (2 ng/ml; R&D Systems) and anti-IgD dextran (Fina Biosolutions) for CSR to IgA. Additonally, LPS was used in combination with, rmIL-4 (5 ng/ml) for CSR to IgG1, and IFN-γ (50 ng/ml; PeproTech Inc.) for CSR to IgG3. Cells were collected on day 4 for surface Ig analysis, after staining with FITC-anti-mouse IgG1 (clone A85-1), anti-mouse IgG2a (clone R19-15), anti-mouse IgG2b (clone R12-3) anti-mouse IgG3 (clone R40-82) or anti-mouse IgA (clone C10-3) rat mAb and PE-anti-mouse CD45R (B220) (clone RA3-6B2) rat mAb (BD Biosciences). Cells were fixed with 1% paraformaldehyde in PBS and analyzed by FACS.

Peyer's patch B cells were stained with phycoerythrin (PE)-Labeled anti-mouse CD45R (B220) ratmAb (RA3-6B2, eBiosciences) and FITC-labeled PNA (E-Y Laboratories, San Mateo, Calif.), 7AAD (Biolegend), and APC-labeled IgM (Biolegend), and analyzed in a FACScalibur (Becton-Dickinson) and FlowJo software.

Real-time PCR can be used to measure the amount of IL40 mRNA in a given sample. A variety of machines are available for this purpose and each requires the design of specific primers in order to amplify and measure the right mRNA. A Lightcycler from Roche (Indianapolis, Ind., U.S.A.) was used.

### EXAMPLE 2

### Identification of Cells Carrying IL40 Receptor

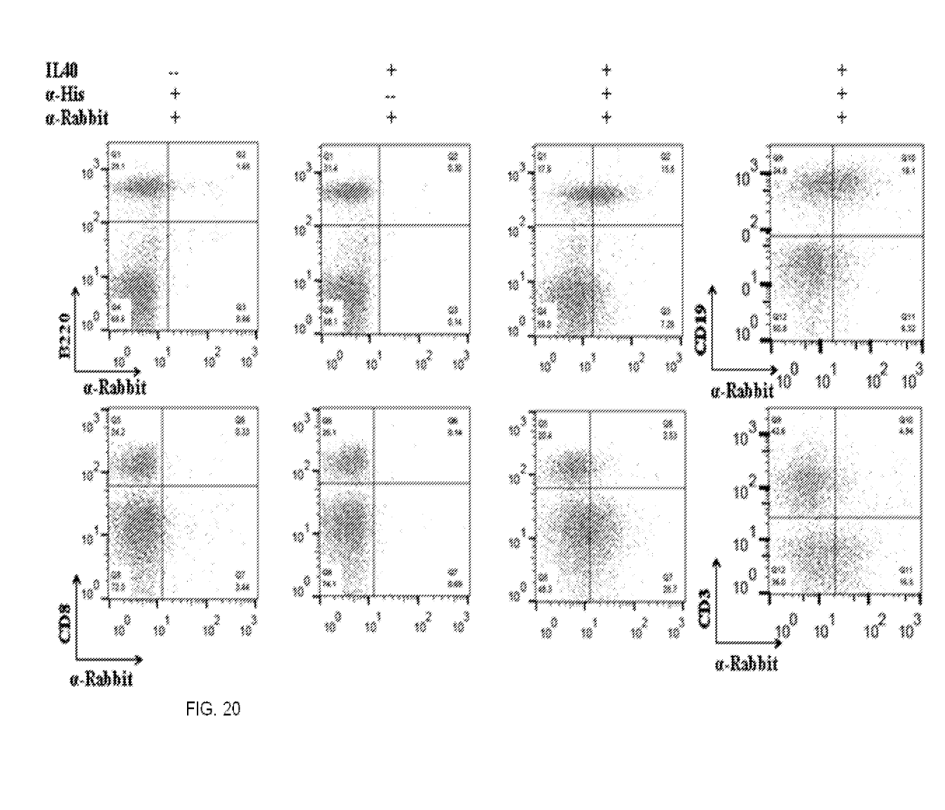

To determine which cell carry IL40 receptor, spleenocytes from C57BL/6 mouse were collected, homogenized and placed into FACS tubes (0.5×10E6 cells per tube). First, spleenocytes were incubated with FACS blocking buffer for 30 min on ice. Then, cells were washed once with ice cold FACS washing buffer and incubated with recombinant His-tagged IL40 (10 ug/ml) for 30 min on ice. After, cells were washed twice and incubated with rabbit anti-6× His tag antibody (1:500 dilution) on ice for 30 min. Then cells were washed twice and incubated with the goat anti-rabbit secondary antibody conjugated to FITC (1:200 dilution), isotype control or other cell surface staining antibodies. Results are show in FIG. 20. This experiment indicated that IL40 binds B cells, but not Tcells.

### Reagents Used for the Experiment:

Rabbit anti-6× His Tag Antibody (Abcam, Catalog Number ab9108)

FITC Donkey anti-Rabbit (Biolegend, catalog numer 406403)

Rabbit IgG isotype control (Santa Cruz, catalog number sc-2027)

PE anti-mouse CD45R/B220 (Biolegend, catalog number 103207)

APC anti-mouse CD8b.2 antibody (Biolegend, catalog number 140409)

PE anti-mouse CD3 antibody (Biolegend, catalog number 100307)

APC anti-mouse CD19 antibody (Biolegend, catalog number 115511)

### EXAMPLE 3

Functional response of receptor expressing cells to cytokine In order to find biomarker genes modulated by IL40 in responding cells, cells from immune organs like spleen, lymph nodes, thymus or bone marrow can be incubated with various doses of recombinant mouse or human IL40 (depending on the origin of cells used) and then incubated for several hours (6-8-24) in order to let the cells transcribe the mRNA of these genes. The cells are then harvested and processed for microarray analyses using genearrays from Affymetrix. The mRNA is prepared using standard techniques and then cDNA is produced for hybridization with the microarrays. The microarrays are read and the data analyzed with proprietary Affymetrix software. The genes controlled by

### EXAMPLE 4

Evaluation of IL40 sequence variants for agonist or antagonist function. The functional responses of receptor-expressing cells can be used to monitor agonist or antagonist function. To this end, the receptor-expressing cells can be incubated with either agonists or antagonists and monitor the expression of IL40-responsive genes by qPCR. The levels of these genes should indicate whether IL40 was able to bind to its receptor or not. The responder cells will incubated with IL40 agonists which should induce the same genes as those induced by IL40 in these responder cells. Antagonists should block the induction of these biomarker genes if the responder cells are incubated with IL40 and each of the candidate antagonists.

### EXAMPLE 5

Prophetic example of identification of receptor structure. The receptors for cytokine can be made up of several protein chains. Established methods can be used that have served well to identify the IL40 receptor, by using several possible methods as described below.

method A: Seed method, cDNA libraries, and test for which clones bind. cDNA libraries can be prepared of cells known to bind IL40. Which cells bind IL40 can be determined by labeling IL40 with radioactivity tags and performing radioimmunoassay. Alternatively, His-tagged IL40 can be used to bind to cells and use an anti-His antibody to detect the binding of the cytokine to the cells that express the receptor. Other tags can be used, like FLAG instead of His. Once cells that express the receptor are identified, cDNA libraries of these cells can be produced, and transfect cells such as HEK293 or BAF3 cells with pools of cDNA library clones. Which of these cells now bind IL40 can be monitored as described above. The fluorescently labeled cells can be sorted by flow cytometry and cultured to use as a source of mRNA. This mRNA can be used to clone the cDNA from the library that was transfected into that given cell, that is also responsible for the expression of the IL40 receptor chains. Specific sequences in the cDNA construct can be used to design primers to PCR out the library cDNAs found in that cell. This method can be used independently of the number of chains that the IL40 receptor is composed of, the only difference would be the frequency with which the positive cells will be identified.

method B: biochemical method, label ligand and isolate binding complexes. The ligand can be labeled with tags like His or FLAG (or others) and incubated with membranes from cells that express the IL40 receptor. Various detergents can be added to optimize the ability of the ligand to bind the receptor and be able to run the complex in a gel following immunoprecipitation with an anti-His or anti-Flag antibody. This method should work regardless of the number of chains that the IL40 receptor has. The resulting immunoprecipitating bands can be isolated and sequenced and bioinformatics can be used to determine the identity of the gene encoding each protein.

method C: genetic method; 2 hybrid system. IL40 interacting proteins can be identified using the yeast two hybrid system. IL40 would be prepared as a fusion protein (for example GALBD) and the potentially interacting proteins can be prepared as fusion proteins labeled with GALAD. In this case, IL40 would be the ‘bait’ and the interacting protein would be the ‘prey’. The system is tested in Saccharomyces cerevisiae, a yeast system where the interacting proteins will cause the transcription of a reported gene (for example, LacZ). Yeast turning on the LacZ gene would be those where successful interaction (complementation) of two proteins has occurred. This system can identify several proteins that make up the IL40 receptor.

### EXAMPLE 6

### Production of Antibodies

method A: mouse or rabbit polyclonals; immunoselection. Antibodies can be produced by immunizing animals with recombinant human, mouse or rat IL40. Sheep, goats, donkeys or horses can be immunized. Several immunization doses can be used and the production of antibodies can be monitored by ELISA. Once the desired response is achieved, the antibodies can be harvested by bleeding the immunized animal and obtaining the serum.

method B: mouse or rat monoclonal antibodies. Monoclonal antibodies can be made against human IL40 by immunizing mice with IL40, monitoring the response of the mice and selecting those that show strong responses against IL40. The spleen is obtained and fused with polyethelene glycol or similar fusing reagent to myelomas cells to produce hybridomas. The resulting hybridomas can be grown in vitro and selected using HAT medium to selective grow hybridomas. The resulting hybridomas are cloned, and the producing antibodies can be screened by ELISA, western, FACS or immunohistochemistry.

Production of antibodies using phage display. Mice are immunized with human IL40 and their spleens are used to create a phage display library. The library is then screened for its ability to bind recombinant IL40. The successful phage display clones are identified and their sequences obtained in order to obtain sequence information of the binding site of the antibody. The resulting antibody can be engineered using molecular biology techniques into appropriate fully human antibodies.

### EXAMPLE 7

### Nucleic Acid Constructs for Expression of Cytokine

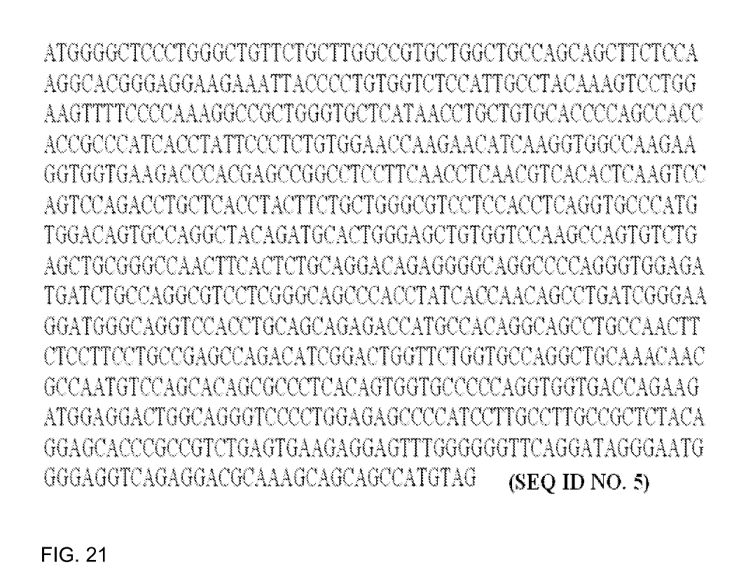

Method 1: cells from human or mice, expressing IL40 mRNA can be used and RT-PCR using anchored oligo-dT can be performed. IL40 cDNA can then be amplified using primers designed to IL40. This cDNA can be inserted into a high expression vector, such as a mammalian, bacterial, or insect plasmid vector. The cDNA containing IL40 gene is inserted into the appropriate recipient cells for each system, and allowed to incubate so the cells can produce the IL40 protein. The supernatant of these cells is harvested and used to purify IL40 using biochemical methods. The human IL40 cDNA sequence (SEQ ID NO. 4) is shown in FIG. 21.

Method 2: Same as method 1, but cloning splice variants of IL40 mRNA by designing primers specific to exons/introns not contained in Method 1.

Method 3: Same as method 1, except sequences encoding a tagged or reporter gene can be inserted, to create fusion proteins. Fusion proteins can be used to detect cells producing Il40. Tagged fusion proteins can also be used for western blotting or affinity purification.

Method 4: Same as method 1, except the promoter can be replaced with a highly inducible, or highly active promoter from a different gene. This can allow us to control the expression of the transgene in the final expression system.

### EXAMPLE 8

### Use of Above Nucleic Acid Constructs to Make and Purify IL40 Protein

Method 1: A plasmid vector construct can be used containing IL40 cDNA insert with a positive or negative selection agent (neomycin/β galactosidase/GFP), to transfect mammalian (HEK, HELA, etc) cell lines, insect cells, or bacteria, depending on which vector the IL40 cDNA was inserted into. The transformed/transfected cells that express the plasmid can be positively or negatively selected for, to eliminate cells that will not produce recombinant protein. High amounts of recombinant IL40 can be obtained directly from supernatant or lysates of the transfected/transformed cells.

Method 2: in a different method, chemical precipitation can be performed on the lysates of cells transfected/transformed with a vector containing IL40 cDNA insert, to obtain concentrated amounts of proteins. Chemical methods include salt, pH, organic solvent, metal ions, non-ionic polymer, methods that non-specifically precipitate all proteins. The lysates containing recombinant protein are mixed with the chemical precipitating agent. The precipitates can be collected by low speed centrifugation or decanting. The precipitates then can be resuspended by dialyzing through a dialysis tube that allows the smaller precipitating agent to pass through the membrane, while keeping the proteins in the tube. The precipitates will contain concentrated amounts total proteins, including the recombinant protein.

Method 3:. In a different method, affinity purification can be performed on the lysates of cells transfected/transformed with a vector containing IL40 cDNA insert using a column containing anti-IL40 antibodies or recombinant IL40 receptor linked to magnetic beads. The column can be washed, to remove non-specific binding of proteins, while maintaining binding of recombinant IL40 to the antibody linked to beads. The column can then be eluted using a change in pH or salt that affects the interaction between rIL40 and the anti-IL40 antibody.

Method 4: In a different method the lysates of cells transfected/transformed with a vector containing IL40 cDNA insert, can be collected and concentrated using size exclusion through a membrane. A centrifuge column can be used that contains a membrane only permeable to proteins less than 30 KD. Proteins less than 30 KD can then be collected, which should include high amounts of recombinant IL40 produced from the transfected/transformed cells.

Method 5: In a different method, the lysates of cells transfected/transformed with a vector containing IL40 cDNA insert can be collected and purified through High performance liquid chromatography. Since each protein interacts differently with the analyte, the fraction containing recombinant IL40 can be collected.

### EXAMPLE 9

### Isolation of IL40 Receptor Expressing Cells (FACS Isolation)

method A: isolation, purification. To determine which cell carry IL40 receptor(s), mouse splenocytes are collected, homogenized and placed into FACS tubes (0.5e6 cells per tube). First, splenocytes are incubated with FACS blocking buffer for 30min on ice. Then, cells are washed once with ice cold FACS washing buffer and incubated with recombinant His-tagged IL40 (10 ug/ml) for 30 min on ice. After, cells are washed twice and incubated with rabbit anti-His tag antibody (1:500 dilution) on ice for 30min. The cells are then washed twice and incubated with goat anti-rabbit secondary antibody conjugated to FITC; isotype control or other cell surface staining antibodies. This experiment indicates that IL40 binds to B cells, which can be used as positive control.

The labeled cells can be sorted by flow cytometry based on their expression of FITC. The sorted cells should express the IL40 receptor.

method B: depletion from cell population. IL40 labeled with a toxin (like an antibody drug conjugate, for example) can be used to destroy cells that express the IL40 receptor. An antibody against the IL40 receptor can also be used for the same purpose, either alone or as an antibody-drug conjugate.

method C: in animal; depletion of cells. Intravenous injection of an anti-IL40 receptor monoclonal antibody can be used to deplete IL40 receptor-expressing cells. The antibody can be a “naked” antibody or an antibody drug conjugate.

### EXAMPLE 10

Use of IL40 transgenic or knockout mouse to discover effects of IL40. An IL40^{-/-} mouse can be used to investigate the function of IL40. This mouse has been published (Tang, T. et al. A mouse knockout library for secreted and trasnmembrane proteins, 2010. Nat. Biotechnol. 7: 749-55). The only phenotype observed was elevated IgG1 in serum when mouse was immunized. Immune tissues from this mouse can be obtained and they can be activated with different agents (anti-CD3 and anti-CD28 for T cells; lipopolysaccharide (LPS) or anti-CD40 and IL4 for B cells; LPS for monocytes). Microarrays analysis can then be performed to identify genes differentially expressed between wild type and IL40^{-/-} tissues. The mouse can also be phenotyped using flow cytometry of various tissues, or perform blood chemistry analyses as well as blood cell counts. The mouse can be used in various models of disease, including cancer, autoimmune diseases, or infectious disease;

Besides the IL40^{-/-} mouse, IL40 can also be overexpressed as a transgene in mice. This would exaggerate the effects of IL40 in vivo and would let investigate the effects of this cytokine in a mouse model. To do this, the transgene is introduced by pronuclear injection into a single cell of the mouse embryo and it is integrated in the genome. The gene is under the control of a promoter that can be triggered to express the gene. Overexpression of IL40 in vivo would lead us to understand the effects of this cytokine in vivo. Phenotyping of the resulting transgenic IL40 mouse would allow us to understand its function in vivo.

### REFERENCES

The following publications are incorporated by reference herein:
1. Oppenheim J J. Cytokines: past, present, and future. International journal of hematology. 2001; 74(1):3-8.
2. Dinarello C A. Historical insights into cytokines European journal of immunology. 2007; 37 Suppl 1:S34-45.
3. Shi Y, Ullrich S J, Zhang J, Connolly K, Grzegorzewski K J, Barber M C, et al. A novel cytokine receptor-ligand pair. Identification, molecular characterization, and in vivo immunomodulatory activity. The Journal of biological chemistry. 2000; 275(25): 19167-76.
4. Zou Q, Wang Z, Guan X, Liu B, Wu Y, Lin Z. An approach for identifying cytokines based on a novel ensemble classifier. BioMed research international. 2013; 2013:686090.
5. Lee J, Hever A, Willhite D, Zlotnik A, Hevezi P. Effects of RNA degradation on gene expression analysis of human postmortem tissues. FASEB journal: official publication of the Federation of American Societies for Experimental Biology. 2005; 19(10):1356-8.
6. Roth R B, Hevezi P, Lee J, Willhite D, Lechner S M, Foster A C, et al. Gene expression analyses reveal molecular relationships among 20 regions of the human CNS. Neurogenetics. 2006; 7(2):67-80.
7. Luu V P, Hevezi P, Vences-Catalan F, Maravillas-Montero J L, White C A, Casali P, et al. TSPAN33 is a novel marker of activated and malignant B cells. Clinical Immunology. 2013; 149(3, Part B):388-99.
8. Ioannidou E. Therapeutic modulation of growth factors and cytokines in regenerative medicine. Current pharmaceutical design. 2006; 12(19):2397-408.
9. Velazquez L, Cheng A M, Fleming H E, Furlonger C, Vesely S, Bernstein A, et al. Cytokine signaling and hematopoietic homeostasis are disrupted in Lnk-deficient mice. The Journal of experimental medicine. 2002; 195(12):1599-611.
10. Clark H F, Gurney A L, Abaya E, Baker K, Baldwin D, Brush J, et al. The secreted protein discovery initiative (SPDI), a large-scale effort to identify novel human secreted and transmembrane proteins: a bioinformatics assessment. Genome research. 2003; 13(10):2265-70.
11. Xu Z, Fulop Z, Wu G, Pone E J, Zhang J, Mai T, et al. 14-3-3 adaptor proteins recruit AID to 5′-AGCT-3′-rich switch regions for class switch recombination. Nature structural & molecular biology. 2010; 17(9): 1124-35.
12. Loignon M, Perret S, Kelly J, Boulais D, Cass B, Bisson L, et al. Stable high volumetric production of glycosylated human recombinant IFNalpha2b in HEK293 cells. BMC biotechnology. 2008; 8:65.
13. Ma A, Koka R, Burkett P. Diverse functions of IL-2, IL-15, and IL-7 in lymphoid homeostasis. Annual review of immunology. 2006; 24:657-79.
14. Rosenberg B R, Papavasiliou F N. Beyond SHM and CSR: AID and related cytidine deaminases in the host response to viral infection. Advances in immunology. 2007; 94:215-44.
15. Lycke N Y, Bemark M. The role of Peyer's patches in synchronizing gut IgA responses. Frontiers in immunology. 2012; 3:329.
16. Fagarasan S, Kinoshita K, Muramatsu M, Ikuta K, Honjo T. In situ class switching and differentiation to IgA-producing cells in the gut lamina propria. Nature. 2001; 413(6856):639-43.
17. Park S R, Zan H, Pal Z, Zhang J, Al-Qahtani A, Pone E J, et al. HoxC4 binds to the promoter of the cytidine deaminase AID gene to induce AID expression, class-switch DNA recombination and somatic hypermutation. Nature immunology. 2009; 10(5):540-50.
18. Tezuka H, Abe Y, Iwata M, Takeuchi H, Ishikawa H, Matsushita M, et al. Regulation of IgA production by naturally occurring TNF/iNOS-producing dendritic cells. Nature. 2007; 448(7156):929-33.
19. Chen L, Chen Z, Baker K, Halvorsen E M, da Cunha A P, Flak M B, et al. The short isoform of the CEACAM1 receptor in intestinal T cells regulates mucosal immunity and homeostasis via Tfh cell induction. Immunity. 2012; 37(5):930-46.
20. Muramatsu M, Kinoshita K, Fagarasan S, Yamada S, Shinkai Y, Honjo T. Class switch recombination and hypermutation require activation-induced cytidine deaminase (AID), a potential RNA editing enzyme. Cell. 2000; 102(5):553-63.
21. Rada C, Di Noia J M, Neuberger M S. Mismatch recognition and uracil excision provide complementary paths to both Ig switching and the A/T-focused phase of somatic mutation. Molecular cell. 2004; 16(2):163-71.
22. Cato M H, Chintalapati S K, Yau I W, Omori S A, Rickert R C. Cyclin D3 is selectively required for proliferative expansion of germinal center B cells. Molecular and cellular biology. 2011; 31(1):127-37.
23. Durandy A, Kracker S, Fischer A. Primary antibody deficiencies. Nature reviews Immunology. 2013; 13(7):519-33.
24. Bergqvist P, Gardby E, Stensson A, Bemark M, Lycke N Y. Gut IgA class switch recombination in the absence of CD40 does not occur in the lamina propria and is independent of germinal centers. Journal of immunology. 2006; 177(11):7772-83.
25. Liu J, Karypis G, Hippen K L, Vegoe A L, Ruiz P, Gilkeson G S, et al. Genomic view of systemic autoimmunity in MRL1pr mice. Genes and immunity. 2006; 7(2):156-68.
26. Pruitt K D, Harrow J, Harte R A, Wallin C, Diekhans M, Maglott D R, et al. The consensus coding sequence (CCDS) project: Identifying a common protein-coding gene set for the human and mouse genomes. Genome research. 2009; 19(7):1316-23.
27. Harte R A, Farrell C M, Loveland J E, Suner M M, Wilming L, Aken B, et al. Tracking and coordinating an international curation effort for the CCDS Project. Database: the journal of biological databases and curation. 2012; 2012:bas008.
28. Gray K A, Daugherty L C, Gordon S M, Seal R L, Wright M W, Bruford E A. Genenames.org: the HGNC resources in 2013. Nucleic acids research. 2013; 41(Database issue):D545-52.
29. Burkhardt A M, Tai KP, Flores-Guiterrez J P, Vilches-Cisneros N, Kamdar K, Barbosa-Quintana O, et al. CXCL17 is a mucosal chemokine elevated in idiopathic pulmonary fibrosis that exhibits broad antimicrobial activity. Journal of immunology. 2012; 188(12):6399-406.
30. Cazac B B, Roes J. TGF-beta receptor controls B cell responsiveness and induction of IgA in vivo. Immunity. 2000; 13(4):443-51.
31. Gros M J, Naquet P, Guinamard R R. Cell intrinsic TGF-beta 1 regulation of B cells. Journal of immunology. 2008; 180(12):8153-8.
32. Klein J, Ju W, Heyer J, Wittek B, Haneke T, Knaus P, et al. B cell-specific deficiency for Smad2 in vivo leads to defects in TGF-beta-directed IgA switching and changes in B cell fate. Journal of immunology. 2006; 176(4):2389-96.
33. Snapper C M, Waegell W, Beernink H, Dasch J R. Transforming growth factor-beta 1 is required for secretion of IgG of all subclasses by LPS-activated murine B cells in vitro. Journal of immunology. 1993; 151(9):4625-36.
34. Wong S C, Oh E, Ng CH, Lam KP. Impaired germinal center formation and recall T-cell-dependent immune responses in mice lacking the costimulatory ligand B7-H2. Blood. 2003; 102(4):1381-8.
35. Good-Jacobson K L, Szumilas C G, Chen L, Sharpe A H, Tomayko M M, Shlomchik M J. PD-1 regulates germinal center B cell survival and the formation and affinity of long-lived plasma cells. Nature immunology. 2010; 11(6):535-42.
36. Gohda M, Kunisawa J, Miura F, Kagiyama Y, Kurashima Y, Higuchi M, et al. Sphingosine 1-phosphate regulates the egress of IgA plasmablasts from Peyer's patches for intestinal IgA responses. Journal of immunology. 2008; 180(8):5335-43.
37. Harris D P, Haynes L, Sayles P C, Duso D K, Eaton S M, Lepak N M, et al. Reciprocal regulation of polarized cytokine production by effector B and T cells. Nature immunology. 2000; 1(6):475-82.
38. Vilcek J, Feldmann M. Historical review: Cytokines as therapeutics and targets of therapeutics. Trends in pharmacological sciences. 2004; 25(4):201-9.
39. Feldmann M. Many cytokines are very useful therapeutic targets in disease. The Journal of clinical investigation. 2008; 118(11):3533-6.
40. Nakamura R M (1983) Monoclonal antibodies: methods and clinical laboratory applications. Clin Physiol Biochem 1: 160-172.
41. Leenaars M, Hendriksen C F (2005) Critical steps in the production of polyclonal and monoclonal antibodies: evaluation and recommendations. ILAR J 46: 269-279.
42. Tomita M, Tsumoto K (2011) Hybridoma technologies for antibody production. Immunotherapy 3: 371-380.
43. Siegel D L (2002) Recombinant monoclonal antibody technology. Transfus Clin Biol 9: 15-22.
44. Glassy M C (1993) Production methods for generating human monoclonal antibodies. Hum Antibodies Hybridomas 4: 154-165.
45. Butler M, Meneses-Acosta A (2012) Recent advances in technology supporting biopharmaceutical production from mammalian cells. Appl Microbiol Biotechnol 96: 885-894.
46. Rasmussen S K, Naested H, Muller C, Tolstrup A B, Frandsen T P (2012) Recombinant antibody mixtures: production strategies and cost considerations. Arch Biochem Biophys 526: 139-145.
47. Marichal-Gallardo P A, Alvarez M M (2012) State-of-the-art in downstream processing of monoclonal antibodies: process trends in design and validation. Biotechnol Prog 28: 899-916.
48. Chon J H, Zarbis-Papastoitsis G (2011) Advances in the production and downstream processing of antibodies. N Biotechnol 28: 458-463.
49. Di Fede G, Bronte G, Rizzo S, Rolfo Cervetto C, Cocorullo G, et al. (2011) Monoclonal antibodies and antibody fragments: state of the art and future perspectives in the treatment of non-haematological tumors. Expert Opin Biol Ther 11: 1433-1445.
50. Chiarella P (2011) Production, novel assay development and clinical applications of monoclonal antibodies. Recent Pat Anticancer Drug Discov 6: 258-267.
51. Kaneko E, Niwa R (2011) Optimizing therapeutic antibody function: progress with Fc domain engineering. BioDrugs 25: 1-11.
52. Sczakiel G (2000) Theoretical and experimental approaches to design effective antisense oligonucleotides. Front Biosci 5: D194-201.
53. Far R K, Leppert J, Frank K, Sczakiel G (2005) Technical improvements in the computational target search for antisense oligonucleotides. Oligonucleotides 15: 223-233.
54. Pascal, L. E., et al., BMC Genomics 2008, 9:246.
55. Gry, M., et al., BMC Genomics 2009, 10:365.
56. Bakhtiyari S, Haghani K, Basati G, Karimfar M H (2013) siRNA therapeutics in the treatment of diseases. Ther Deliv 4: 45-57.
57. Peek A S, Behlke M A (2007) Design of active small interfering RNAs. Curr Opin Mol Ther 9: 110-118.
58. Baranova A, Bode J, Manyam G, Emelianenko M (2011) An efficient algorithm for systematic analysis of nucleotide strings suitable for siRNA design. BMC Res Notes 4: 168.
59. Juliano, R. et al., Nucleic Acids Res. 2008 July; 36(12): 4158-4171.

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the invention and the following claims.

What is claimed is:

1. An antibody against the C17orf99 polypeptide gene product (IL40), wherein the antibody is:
a) an IgG, IgM, IgA, IgD or IgE;
b) a monoclonal antibody;
c) an Fab′, Fab, F(ab′)₂, single domain antibody (sdAb), Fv, or scFv (single chain Fv);
d) a labeled antibody;
e) a neutralizing antibody; or
f) any combination of a)-e).

2. A method of detecting IL40 in a sample, comprising immunodetecting IL40 using the antibody of claim 1 as a detection agent in an immunodetection method.

3. The method of claim 2, wherein the immunodetection method is enzyme-linked immunosorbent assay (ELISA), histology, fluorescence-activated cell sorting, radioimmunoassay (RIA), immunoradiometric assay, immunohistochemistry, fluoroimmunoassay, chemiluminescent assay, bioluminescent assay, Western blotting, or dot blotting.

4. A method of treating a disease involving IL40 in a subject in need thereof, comprising administering to the subject a therapeutically effective amount of an antibody of claim 1 to neutralize IL40.

5. The method of claim 4, wherein the disease is an autoimmune disease or lymphoma.

6. A method of detecting IL40 in a sample, comprising immunodetecting IL40 using the antibody of claim 1 as a detection agent in a diagnostic or theranostic method for a disease involving IL40.

7. The method of claim 6, wherein the disease is lymphoma, leukemia, immunodeficiency or an autoimmune disease.

8. The method of claim 7, wherein the autoimmune disease is systemic lupus erythematosus, rheumatoid arthritis, or psoriasis, and the immunodeficiency is IgA deficiency syndrome.

9. A peptide or isolated protein comprising a part of or the entire amino acid sequence of IL40 or mature form of IL40.

10. The peptide or protein of claim 9, wherein the peptide or protein is:
a) a sequence variant, polymorphism, or species counterpart of IL40;
b) a substitutional, insertional, or deletion variant of IL40;
c) a non-sequence derivative of IL40, selected from the group consisting of glycosylation modified IL40, chemically modified IL40, and a conjugate of IL40;
d) a functional variant of IL40;
e) a functional segment of IL40, a conserved region of IL40, or a non-conserved region of IL40;
f) a fusion protein of IL40; or
g) any combination of a)-f).

11. A method of treating a disease involving IL40 in a subject in need thereof, comprising administering to the subject a therapeutically effective amount of the peptide or protein of claim 9 or 10, wherein the peptide or protein is an IL40 antagonist.

12. The method of claim 11, wherein the disease is autoimmune disease or lymphoma.

13. A method of treating a disease involving IL40 in a subject in need thereof, comprising administering to the subject a therapeutically effective amount of IL40, or the peptide or protein of claim 9 or 10, wherein the peptide or protein is an IL40 agonist.

14. The method of claim 13, where the disease is IgA deficiency syndrome, Hodgkin or non Hodgkin Lymphomas, diffuse large cell lymphoma, mycosis fungoides, mantle cell lymphoma, multiple myeloma, or another lymphoma or leukemia; rheumatoid arthritis, systemic lupus erythematosus, Sjogren's syndrome, Hashimoto thyroiditis, scleroderma, Graves' disease, Crohn's disease, ulcerative colitis, primary biliary cirrhosis, autoimmune hepatitis, multiple sclerosis, psoriasis, atopic dermatitis, idiopathic pulmonary fibrosis, hypersensitivity pneumonitis, non-specific interstitial pneumonia, or another autoimmune disease.

15. A method of diagnosing a disease involving IL40, comprising using the peptide or protein of claim 9 or 10 as a target or sample control in a diagnostic/theranostic method.

16. The method of claim 15, wherein the disease is lymphoma, an autoimmune disease, systemic lupus erythematosus, rheumatoid arthritis, or psoriasis.

17. A method of purifying or isolating a subset of cells that express IL40, comprising using the antibody of claim 1 as a purification/isolating agent to purify or isolate the subset of cells.

18. The method of claim 17, wherein the subset is purified or isolated by fluorescence-activated cell sorting (FACS) to select the cell subsets.

19. A cell producing the antibody of claim 1, wherein the cell is a hybridoma, a recombinant bacterial cell, a recombinant yeast cell, or a recombinant mammalian cell.

20. An animal comprising the cell of claim 19.

21. The peptide or protein of claim 10, wherein the functional variant is an agonist or antagonist of IL40, and the fusion protein is a covalent or noncovalent construct, or a labeled construct.

22. A method of inducing an immune cell, comprising using the peptide or protein of claim 9 or 10 as an active agent to induce the immune cell to produce synaptogyrin 2 and/or other IL40-induced proteins produced by B cells, or to induce differentiation or maturation of the immune cell.

23. A method of identifying a receptor for IL40, comprising using the peptide or protein of claim 9 or 10 as a ligand for binding to the IL40 receptor.

24. A cell producing the peptide or protein of claim 9 or 10, wherein the cell is a recombinant bacterial cell, a recombinant yeast cell, or a recombinant mammalian cell.

25. An animal comprising the cell of claim 24.

26. A nucleic acid comprising a part of or the entire nucleotide sequence of an IL40 gene or IL40 cDNA.

27. A nucleic acid comprising a part of or the entire nucleotide sequence of an IL40 gene or IL40 cDNA, wherein the nucleic acid encodes a peptide or protein of claim 9.

28. The nucleic acid of claim 26 or 27, wherein the nucleic acid is:
a) conjugated to another nucleotide sequence, to a label, or to a chemical derivative;
b) a primer, a probe, an antisense molecule, or an oligonucleotide based on the IL40 gene or IL40 cDNA sequence;
c) a recombinant construct attached to a heterologous nucleic acid sequence; or
d) any combination of a)-c).

29. The nucleic acid of claim 28, wherein the heterologous nucleic acid sequence is a promoter, an enhancer, a vector, or an expression vector.

30. A method of treating a disease involving IL40 in a subject in need thereof, comprising administering to the subject a therapeutically effective amount of the nucleic acid of claim 26 or 27, wherein the nucleic acid decreases IL40 expression.

31. The method of claim 30, wherein the disease is an autoimmune disease or lymphoma.

32. A method of treating a disease involving IL40 in a subject in need thereof, comprising administering to the subject a therapeutically effective amount of the nucleic acid of claim 26 or 27, wherein the nucleic acid increases IL40 expression.

33. The method of claim 32, wherein the disease is IgA deficiency syndrome, Hodgkin or non Hodgkin Lymphomas, diffuse large cell lymphoma, mycosis fungoides, mantle cell lymphoma, or another lymphoma or leukemia; rheumatoid arthritis, systemic lupus erythematosus, Sjogren's syndrome, Hashimoto thyroiditis, scleroderma, Graves' disease, Crohn's disease, ulcerative colitis, primary biliary cirrhosis, autoimmune hepatitis, multiple sclerosis, psoriasis, atopic dermatitis, idiopathic pulmonary fibrosis, hypersensitivity pneumonitis, non-specific interstitial pneumonia, or another autoimmune disease.

34. The method of claim 30, wherein the nucleic acid is an RNAi molecule.

35. A method of diagnosing a disease involving IL40, comprising probing a sample by using the nucleic acid of claim 26 or 27 as a probe in a diagnostic/theranostic method for the disease.

36. The method of claim 35, wherein the disease is lymphoma, autoimmune disease, systemic lupus erythematosus, rheumatoid arthritis, or psoriasis.

37. A cell comprising a recombinant form of the nucleic acid of claim 26 or 27, wherein the cell is a recombinant bacterial cell, a recombinant yeast cell, or a recombinant mammalian cell.

38. An animal comprising the cell of claim 37.

39. A method of selecting a subset of cells expressing IL40, comprising
adding a molecule that binds IL40 to a cell population comprising cells expressing IL40, and
selecting cells labeled with the IL40 binding molecule to provide a population of selected cells.

40. The method of claim 39, wherein:
a) the cells expressing IL40 are mouse, rat or human cells;
b) the IL40 binding molecule is an anti-IL40 antibody or an IL40 receptor;
c) the selected cells are selected from blood, body fluids, cell suspensions or patient samples;
d) the selected cells are research tools for studying IL40 expressing cells; or
e) the selected cells are blood cells and are:
i) a source of mRNA for immunoglobulins produced by the selected cells;
ii) a source of new methods for producing fully humanized antibodies; or
f) any combination of a)-e).

41. A method of treating a disease involving IL40 in a subject in need thereof, comprising administering to the subject a therapeutically effective amount of the selected cells of claim 39.

42. The method of claim 41, wherein the disease is IgA deficiency syndrome, Hodgkin or non Hodgkin Lymphomas, diffuse large cell lymphoma, mycosis fungoides, mantle cell lymphoma, or another lymphoma or leukemia; rheumatoid arthritis, systemic lupus erythematosus, Sjogren's syndrome, Hashimoto thyroiditis, scleroderma, Graves' disease, Crohn's disease, ulcerative colitis, primary biliary cirrhosis, autoimmune hepatitis, multiple sclerosis, psoriasis, atopic dermatitis, idiopathic pulmonary fibrosis, hypersensitivity pneumonitis, non-specific interstitial pneumonia, or another autoimmune disease.

43. A method of detecting activated B-cells in a subject, comprising measuring the level of IL40 in the subject, wherein an increased level of IL40 over control is indicative of activated B-cells.

44. The method of claim 43, wherein:
a) the IL40 level is measured by an immunodetection technique;
b) the method further includes measuring another biomarker;
c) the method diagnoses autoimmunity or lymphoma in the subject in need of such diagnosis, wherein the increased level is indicative of lymphoma or autoimmunity;
d) the increased level of IL40 defines an IL40-producing subtype of lymphoma or autoimmune disease; or
e) any combination of a)-d).

45. The method of claim 44, wherein the immunodetection technique is ELISA, histology, fluorescence-activated cell sorting, radioimmunoassay (RIA), immunoradiometric assay, immunohistochemistry, fluoroimmunoassay, chemiluminescent assay, bioluminescent assay, Western blotting or dot blotting.

46. A method of treating lymphoma or autoimmune disease in a subject in need thereof, comprising administering a therapeutically effective amount of an antibody of claim 1, or a nucleic acid of claim 26 or 27, to the subject, or to a tumor, tissue or cell of the subject.

47. The method of claim 46, wherein the antibody is a neutralizing antibody, and the nucleic acid is an antisense RNA.

48. The method of claim 47, wherein the antisense RNA is an RNAi molecule.

49. A method of identifying IL40-producing cells in a subject, comprising using an antibody of claim 1, or a nucleic acid of claim 26 or 27, as a probe to identify the cells.

50. The method of claim 49, wherein the method comprises immunohistochemistry or in situ hybridization.

51. A method of identifying an IL40 receptor, comprising
a) labeling an IL40-responsive cell with IL40, a labeled IL40, a His-tagged IL40, or a combination thereof, and isolating, purifying and/or separating the labeled cell;
b) labeling an IL40-responsive cell with IL40, a labeled IL40, a His-tagged IL40, or a combination thereof, and isolating, purifying and/or separating the labeled cell, wherein the cell is a eukaryotic or bacterial cell expressing the IL40 receptor;
c) identifying a protein that binds to IL40 in a yeast two-hybrid system; or
d) immunoprecipitating an IL40-binding protein from a membrane preparation from cells expressing the IL40 receptor.

52. A method of using IL40, comprising exposing immune cells to IL40 or to a functionally active variant thereof so as to:
a) promote growth and differentiation of B cells in vitro or in vivo;
b) increase growth of a hybridoma culture;
c) increase antibody production by a hybridoma culture; or
d) determine species origin of a tissue or cell by using IL40 from different mammalian species.

53. The method of claim 52, wherein the mammalian species is human, dog, cat, cow, horse, pig, goat, or sheep.

54. The subject matter of any one of claims 1-53 involving autoimmunity, autoimmune disease or lymphoma,
wherein the autoimmune disease is systemic lupus erythematosus, rheumatid arthritis, psoriasis, Graves' disease, autoimmune hepatitis, primary biliary cirrhosis, Hashimoto's thyroiditis, or Sjögren's syndrome, and
the lymphoma is Hodgkin's and non-Hodgkin's lymphoma, mantle cell lymphoma, diffuse large B cell lymphoma, follicular lymphoma, chronic lymphocytic leukemia, MALT lymphoma, Burkitt's lymphoma, mycosis fungoides, or multiple myeloma.

55. The subject matter of any one of claims 1-53 involving treating or treatment,
wherein the antibody, peptide, protein or nucleic acid is delivered locally or systemically.

56. The subject matter of any one of claims 1-53 involving diagnostic, diagnosis, diagnose, or diagnostic/theranostic methods,
wherein the method is practiced on a sample.

57. The subject matter of claim 56, wherein the sample is serum, blood, a body fluid, a tumor, a tissue or a cell.

58. The subject matter of any one of claims 1-53 involving antibody, peptide, protein or nucleic acid molecules, wherein the molecules are in a pharmaceutical formulation that:
a) comprises a pharmaceutically acceptable carrier, excipient, or a combination thereof;
b) is used as a sterile formulation;
c) comprises another therapeutic agent for treating autoimmune disease or lymphoma;
d) is in a slow or sustained release formulation;
e) is in a targeted administration form; or
f) any combination of a)-e).

59. The subject matter of claim 58, wherein the slow or sustained release formulation comprises an emulsion or micelles, and the targeted administration form is a liposome, inclusion complex, or a carrier.


SEQUENCE LISTING (5 sequences)

SEQ ID NO: 1 - protein, 265 aa, Homo sapiens
MGLPGLFCLAVLAASSFSKAREEEITPVVSIAYKVLEVFPKGRWVLITCCAPQPPPPITYSLCGTKNIKVAKKVVKTHEPASFNLNVTLKSSPDLLTYFCWASSTSGAHVDSARLQMHWELWSKPVSELRANFTLQDRGAGPRVEMICQASSGSPPITNSLIGKDGQVHLQQRPCHRQPANFSFLPSQTSDWFWCQAANNANVQHSALTVVPPGGDQKMEDWQGPLESPILALPLYRSTRRLSEEEFGGFRIGNGEVRGRKAAAM

SEQ ID NO: 2 - protein, 190 aa, Homo sapiens
EEITPVVSIAYKVLEVFPKGRWVLITCCAPQPPPPITYSLCGTKNIKVAKKVVKTHEPASFNLNVTLKSSPDLLTYFCWASSTSGAHVDSARLQMHWELWSKPVSELRANFTLQDRGAGPRVEMICQASSGSPPITNSLIGKDGQVHLQQRPCHRQPANFSFLPSQTSDWFWCQAANNANVQHSALTVVP

SEQ ID NO: 3 - protein, 190 aa, Mus musculus
EEQTEGITIAYKVLEVYPQSRRVLITCDAPEASQPITYSLLASRGILVAKKVVHDSVPASFNINITIKSSPDLLTYSCQATSNSGTYGPSSRLQMYQELWAKPVSQLQADFVLRHGDSGPTVELSCLASSGSPPITYRLVGNGGRVLAQQRPLHGKPANFSLPLSQTTGWFQCEAENDVGVDSSARIPLP

SEQ ID NO: 4 - protein, 109 aa, Artificial Sequence
  Consensus sequence
EETIAYKVLEVPRVLITCAPPITYSLIVAKKVVPASFNNTKSSPDLLTYCASSGSRLQMELWKPVSLAFLGPVECASSGSPPITLGGVQQRPHPANFSSQTWFCANSAP

SEQ ID NO: 5 - DNA, 798 bp, Homo sapiens
atggggctccctgggctgttctgcttggccgtgctggctgccagcagcttctccaaggcacgggaggaagaaattacccctgtggtctccattgcctacaaagtcctggaagttttccccaaaggccgctgggtgctcataacctgctgtgcaccccagccaccaccgcccatcacctattccctctgtggaaccaagaacatcaaggtggccaagaaggtggtgaagacccacgagccggcctccttcaacctcaacgtcacactcaagtccagtccagacctgctcacctacttctgctgggcgtcctccacctcaggtgcccatgtggacagtgccaggctacagatgcactgggagctgtggtccaagccagtgtctgagctgcgggccaacttcactctgcaggacagaggggcaggccccagggtggagatgatctgccaggcgtcctcgggcagcccacctatcaccaacagcctgatcgggaaggatgggcaggtccacctgcagcagagaccatgccacaggcagcctgccaacttctccttcctgccgagccagacatcggactggttctggtgccaggctgcaaacaacgccaatgtccagcacagcgccctcacagtggtgcccccaggtggtgaccagaagatggaggactggcagggtcccctggagagccccatccttgccttgccgctctacaggagcacccgccgtctgagtgaagaggagtttggggggttcaggatagggaatggggaggtcagaggacgcaaagcagcagccatgtag